(19) United States
(12) Patent Application Publication
(10) Pub. No.: US 2007/0072238 A1
(43) Pub. Date: Mar. 29, 2007

(54) Wnt-frizzled chimera

(75) Inventor: Ramesh A. Bhat, King Of Prussia, PA (US)

Correspondence Address: DARBY & DARBY P.C., P.O. BOX 5257, NEW YORK, NY, 10150-5257, US

(73) Assignee: Wyeth, Madison, NJ (US)

(21) Appl. No.: 11/515,500

(22) Filed: Sep. 1, 2006

Related U.S. Application Data
(60) Provisional application No. 60/722,890, filed on Sep. 30, 2005.
(60) Provisional application No. 60/720,952, filed on Sep. 26, 2005.

(51) Int. Cl. G01N 33/53; C07H 21/04; C12P 21/06; C07K 14/705

(52) U.S. Cl. 435/7.1; 435/69.1; 435/320.1; 435/325; 530/350; 536/23.5

(57) ABSTRACT

The present invention relates to a Wnt-Frizzled chimera. The present invention also relates to pharmaceutical compositions that can be screened or developed using Wnt-Frizzled chimeras. The methods and pharmaceutical compositions of the present invention can be used to treat bone disorders and cancer.

### CROSS-REFERENCE TO RELATED APPLICATIONS

Priority is claimed under 35 U.S.C. § 119(e) to U.S. Provisional Application Ser. No. 60/720,952, filed on Sep. 26, 2005 and to U.S. Provisional Application Ser. No. 60/722,890, filed on Sep. 30, 2005. The contents of these priority applications are incorporated into the present disclosure by reference and in their entireties.

### FIELD OF THE INVENTION

The present invention relates to Wnt-Frizzled chimeras. The present invention also relates to screening methods and other uses of the Wnt-Frizzled chimeras. The products and methods of the present invention can be used to investigate and develop treatments for bone disorders and cancer.

### BACKGROUND OF THE INVENTION

The topic of bone formation regulation and bone-related disorders has gained considerable attention. For example, in the women's health area there has been a particular focus on the bone-related disorder osteoporosis. Throughout life, there is a constant remodeling of skeletal bone. Bone is formed and maintained by two cell types: osteoblasts that synthesize and mineralize the bone matrix, and osteoclasts that resorb the calcified tissue (Komm and Bodine (2001) in Osteoporosis. Marcus et al. eds. Academic Pres: San Diego, pages 305-337; Bodine and Komm (2002) Vitam. Horm. 64:101-151; Goltzman (2002) Nat. Rev. Drug Discov. 1:784-796). Osteoblasts arise from multipotent mesenchymal stem cells that are located in bone marrow (Lian et al. (1999) in Primer on the metabolic bone diseases and disorders of mineral metabolism. M. J. Favus, ed. Lippincott Williams & Wilkins: Philadelphia, pages 14-29; Bodine and Komm, (2002) Vitam. Horm. 64:101-151; Goltzman (2002) Nat. Rev. Drug Discov. 1:784-796), while osteoclasts originate from hematopoietic bone marrow cells (Teitelbaum (2000) Science 289:1504-1508; Goltzman (2002) Nat. Rev. Drug Discov. 1:784-796). Osteoblast and osteoclast cells work together in a process known as bone remodeling, which is the mechanism by which immature, damaged, or aged bone is replaced with new lamellar bone (Mundy (1999) in Primer on the metabolic bone diseases and disorders of mineral metabolism. Favus, ed. Lippincott Williams & Wilkins: Philadelphia, pages 30-38). Bone remodeling is initiated by recruitment and activation of osteoclasts that remove the mineralized matrix. The process ends about 6 months later with the filling-in of the resorption pit with newly formed osteoid by the osteoblasts. At the end of this last phase, the bone-forming cells experience one of three fates (Manolagas (2000) Endocr. Rev. 21:115-137; Bodine and Komm, (2002) Vitam. Horm. 64:101-151; Goltzman (2002) Nat. Rev. Drug Discov. 1:784-796). They can differentiate to osteocytes upon entrapment within the mineralized matrix; they can differentiate to quiescent lining cells; or they can undergo apoptosis.

The majority of studies on age-related changes in human bone have been directed toward elucidating changes in bone on a morphological level or by quantitatively comparing rates of bone loss. Disruption of the fine balance between the differentiation of new osteoclast and osteoblast cells and the timing of cell death by apoptosis is thought to be an important mechanism behind bone loss disorders. Therapeutic agents that alter the prevalence of apoptosis in osteoblasts and/or osteoclasts are useful and desirable to correct the imbalance in cell numbers that is the basis of diminished bone mass and increased risk of fractures in osteoporosis (for review, see, Manolagas (2000) Endocr. Rev. 21:115-137; Weinstein and Manolagas (2000) Am. J. Med. 108:153-164).

### The Wnt Gene Family

One group of genes and the proteins encoded by them that play an important role in regulating cellular development is the Wnt family of glycosylated lipoproteins. Wnt proteins are a family of growth factors consisting of more than a dozen structurally related molecules that are involved in the regulation of fundamental biological processes such as apoptosis, embryogenesis, organogenesis, morphogenesis, and tumorigenesis (reviewed in Nusse and Varmus (1992) Cell 69:1073-1087). These polypeptides are multipotent factors and have similar biological activities to other secretory proteins such as transforming growth factor (TGF)-β, fibroblast growth factors (FGFs), nerve growth factor (NGF), and bone morphogenetic proteins (BMPs). Members of the Wnt family related to bone include Wnt3 (human sequence set forth in SEQ ID NO: 1), Wnt1 (human sequence set forth in SEQ ID NO: 2), and Wnt10b. Wnt10b endogenously regulates bone formation by increasing bone mass and bone strength, conferring resistance to the loss of bone associated with aging, protecting against bone loss due to ovariectomy, and stimulating osteoblastogenesis (Bennett et al. (2005) Proc. Natl. Acad. Sci. USA 102:3324-3329).

### The Frizzled Family of Proteins

Studies indicate that certain Wnt proteins interact with a family of proteins named “Frizzled” (or “Fz,” “Fzd,” or “FZD”) that act as receptors for Wnt proteins or as components of a Wnt receptor complex (reviewed in Moon et al. (1997) Cell 88:725-728; Barth et al. (1997) Curr. Opin. Cell Biol. 9:683-690). Frizzled proteins contain an amino terminal signal sequence for secretion, a cysteine-rich domain (CRD) that is thought to bind Wnt, seven putative transmembrane domains that resemble a G-protein coupled receptor, and a cytoplasmic carboxyl terminus.

The discovery of the first secreted frizzled-related protein (SFRP) was reported by Hoang et al. ((1996) J. Biol. Chem. 271:26131-26137). This protein, which was called “Frzb” for frizzled motif in bone development, was purified and cloned from bovine articular cartilage extracts based on its ability to stimulate in vivo chondrogenic activity in rats. The human homologue of the bovine gene was also cloned. However, unlike the frizzled proteins, Frzb did not contain a serpentine transmembrane domain. Thus, this new member of the frizzled family appeared to be a secreted receptor for Wnt. The Frzb cDNA encoded for a 325 amino acid/36,000 Dalton (Da) protein that was predominantly expressed in the appendicular skeleton. The highest level of expression was in developing long bones and corresponded to epiphyseal chondroblasts; expression then declined and disappeared toward the ossification center.

The SFRP family of proteins are ˜32-40 kiloDalton (kDa) glycoproteins that were identified as antagonists of Wnt signaling (Rattner et al. (1997) Proc. Natl. Acad. Sci. USA 94:2859-63; Melkonyan et al. (1997) Proc. Natl. Acad. Sci. USA 94:13636-41; Finch et al. (1997) Proc. Natl. Acad. Sci. USA 94:6770-5; Uren et al. (2000) J. Biol. Chem. 275:4374-82; Kawano et al. (2003) J. Cell. Sci. 116:2627-34). In mammals, there are five SFRPs, grouped into two subfamilies based on sequence homology. SFRP-1 (human sequence set forth in SEQ ID NO: 3) is most closely related to SFRP-5 (human sequence set forth in SEQ ID NO: 4) and SFRP-2 (human sequence set forth in SEQ ID NO: 5) (56% and 36% amino acid similarity respectively) and is more distantly related to SFRP-3 (human sequence set forth in SEQ ID NO: 6) and SFRP-4 (human sequence set forth in SEQ ID NO: 7) (19% and 17% amino acid similarity respectively). The SFRPs contain three structural units: an amino terminal signal peptide, a Frizzled (Fzd) type cysteine-rich domain (CRD), and a carboxy-terminal netrin domain. The CRD spans ˜120 amino acids, contains 10 conserved cysteine residues and has 30-50% sequence similarity to the CRD of Fzd receptors. The disulphide linkage and the cysteine spacing of human SFRP-1 has been determined, and the cysteine spacing of the CRD is highly conserved throughout the homologs and orthologs (Chong et al. (2002) J. Biol. Chem. 277:5134-44). Crystallographic data of the CRD of mouse SFRP-3 and mFzd 8 have been resolved, and the structures have revealed the potential for the CRD to homodimerize or heterodimerize between SFRP and Fzd (Dann et al. (2001) Nature 412:86-90). Several biochemical studies have shown the interaction of the SFRP CRD and Wnt and also the complex formation of the CRDs of Fzd and SFRPs (Bafico et al. (1999) J. Biol. Chem. 274:16180-16187). Such findings suggest that SFRP inhibition of Wnt signaling may operate through at least two mechanisms: (i) by competition with Fzd for Wnt ligands, or (ii) in a dominant-negative fashion by direct formation of non-signaling inactive complexes with Fzd receptors (Bafico et al. (1999) J. Biol. Chem. 274:16180-16187; Jones et al. (2002) BioEssays 24:811-820).

The carboxyl-terminal half of SFRPs contains a domain that shares some sequence similarity with the axon guidance protein, netrin (Serafini et al. (1994) Cell 78:409-24). This netrin domain is defined by six cysteine residues and several conserved segments of hydrophobic residues and secondary structures. Such a structural domain has also been found in tissue inhibitors of metalloproteinases, Type1 procollagen C-proteinase enhancer proteins, and complement proteins C3, C4, and C5 (Banyai et al. (1999) Protein Sci. 8:1636-42). The netrin domain in SFRP-1 and SFRP-5 contains a highly charged hyaluronan-binding domain that is responsible for the heparin-binding properties of the protein (Uren et al. (2000) J. Biol. Chem. 275:4374-82). The hyaluronan binding region is shown to be involved in the interaction of SFRP-1 with Wg (Wingless, the Drosophila ortholog of mammalian Wnt-1; Uren et al. (2000) J. Biol. Chem. 275:4374-82).

The biological activity of SFRPs is largely attributed to their role as regulators of Wnt function. Several studies have suggested a role in the regulation of apoptosis (Melkonyan et al. (1997) Proc. Natl. Acad. Sci. USA 94:13636-41; Chong et al. (2002) J. Biol. Chem. 277:5134-44; Han et al. (2004) J. Biol. Chem. 279:2832-2840). In a knockout mouse model, deletion of mouse SFRP-1 led to decreased osteoblast and osteocyte apoptosis, increased osteoprogenitor differentiation, enhanced bone formation and elevated bone mineral density (Bodine et al. (2004) Mol. Endocrinol. 18:1222-37). Thus, some SFRPs have been identified as “SARPs” for secreted apoptosis related proteins. The five known human SFRP/SARP genes are SFRP-1/FrzA/FRP-1/SARP-2, SFRP-2/SDF-5/SARP-1, SFRP-3/Frzb-1/FrzB/Fritz, SFRP-4 and SFRP-5/SARP-3 (Leimeister et al. (1998) Mech. Dev. 75:29-42).

Using a phage display library, a peptide motif that bound to SFRP-1 has been identified (L/V-VDGRW-L/W) (SEQ ID NO: 8) (Chuman et al. (2004) Peptides 25:1831-8) and the interaction of SFRP-1 with RANKL that contained the peptide motif has been demonstrated. Such an interaction of SFRP-1 and RANKL led to the inhibition of osteoclast formation (Hausler et al. (2004) J. Bone Miner. Res. 19:1873-81). Thus the biological role of SFRP-1 has expanded into new avenues beyond its role as a regulator of Wnt action.

### SFRPs and Bone Formation

SFRP-1 and the Wnt signaling pathway have been found to be involved in the regulation of bone formation (Westendorf et al. (2004) Gene 341:19-29). Inhibition of SFRP-1 promotes increased rate of bone formation, a decrease in osteoblast and osteoclast apoptosis, and an increase in osteoblast differentiation. (For review, see, Bodine et al. (2004) Mol. Endocrinol. 18:1222-37.)

hOB SFRP-1 (SEQ ID NO: 9) is regulated by osteogenic or bone-forming agents in human osteoblast (hOB) cell lines in vitro. The expression of this gene is upregulated during hOB differentiation, suggesting it may be involved in the bone formation process. DNA sequence analysis indicated that this gene fragment (SEQ ID NO: 10) shares significant sequence identity to a mouse cDNA called secreted frizzled-related protein (SFRP)-1 (Rattner et al. (1997) Proc. Natl. Acad. Sci. USA 94:2859-2863). Subsequent cDNA cloning and additional sequence analysis indicated that the gene, which is referred to as the hOB SFRP, was, except for a one amino acid difference at position 174, identical to human SFRP-1/FRP-1/SARP-2 (U.S. patent application Ser. No. 10/169,545, incorporated herein by reference in its entirety; Finch et al. (1997) Proc. Natl. Acad. Sci. USA 94:6770-6775; Melkonyan et al. (1997) Proc. Natl. Acad. Sci. USA 94:13636-13641). The Wnt antagonist activity of hOB SFRP-1 was found to have no significant difference to the published human SFRP-1.

Development of an SFRP-1 −/− mouse line provided an experimental approach to address the contribution of Wnt signaling to bone biology and to determine if SFRP-1 regulates osteoblast and osteocyte viability in vivo (see U.S. Patent Application Pub. No. 2004/0115195, U.S. Ser. No. 10/666,851, incorporated herein by reference in its entirety). These mice show that deletion of SFRP-1 not only reduces osteoblast and osteocyte apoptosis, but also potentiates osteoprogenitor cell differentiation and increases trabecular bone formation. Targeted deletion of SFRP-1 delays the onset of age-dependent trabecular bone loss, while having little effect on fertility, body weight, blood and urine chemistries, non-skeletal organs or cortical bone. These results indicate that SFRP-1 not only plays a role in the attainment of peak bone mass, but also regulates senile bone loss.

As further disclosed in U.S. Patent Application Pub. No. 2004/0115195 (U.S. Ser. No. 10/666,851), Wnt prolongs the life of human osteoblasts in vitro, and antagonism of Wnt signaling by hOB SFRP-1 promotes cell death. Also, deletion of SFRP-1 in mice results in increased trabecular bone formation, decreased osteoblast and osteocyte apoptosis, enhanced osteoprogenitor differentiation, and enhanced bone marrow-derived osteoprogenitor cell and calvarial-derived osteoblast proliferation without altering bone resorption or skeletal development. Thus, an inhibitor of SFRP-1 function may increase osteoblast/pre-osteocyte survival and therefore enhance bone formation in vivo.

A need exists for the definitive identification of targets for the treatment of bone disorders (including bone formation disorders and bone density disorders) and degenerative bone disorders, including osteodegeneration disorders (osteopenia, osteoarthritis, and osteoporosis).

### Wnts and Cancer

SFRP-1 and the Wnt signaling pathway also have been found to be associated with cancer. This includes colorectal cancer (Suzuki et al. (2004) Nat. Genet. 36:417-422; Suzuki et al. (2002) Nat. Genet. 31:141-149), breast cancer (Klopocki et al. (2004) Int. J. Oncol. 25:641-9), and leukemia (Lu et al. (2004) Proc. Natl. Acad. Sci. USA 101:3118-3123). Suzuki et al. ((2002) Nat. Genet. 31:141-149) identifies the SFRP family of genes to be preferentially hypermethylated in colorectal cancer. Hypermethylation of the genes prevents transcription; therefore, SFRP expression is reduced or eliminated, allowing Wnt to freely interact with Fz, which in turn allows constitutive signaling of the Wnt pathway. Suzuki et al. ((2004) Nat. Genet. 36:417-422) shows restoration of SFRP function attenuates Wnt signaling and initiates apoptosis in colorectal cancer cells.

### Wnt-Fzd Chimera

The Frizzled receptors act as a co-receptor for Wnt ligands. All frizzled receptors contain an N-terminal cysteine rich domain (CRD) that is homologous to the CRD domain of SFRPs. In particular, a tyrosine residue is conserved in all frizzled receptors and also in SFRP-1, -2, -5 and is replaced by tryptophan in SFRP-3 and -4. Mutation of tyrosine in SFRP-1 results in reduced activity, indicating its importance in the Wnt antagonist function of SFRPs.

Wnt-Fzd chimeras have been studied in Holmen et al. (2002) J. Biol. Chem. 277:34727-34735 and Cong et al. (2004) Development 131:5103-5115. However, there remains a need in the art to understand Wnt signaling. The present invention addresses that need.

### SUMMARY OF THE INVENTION

In one embodiment, the present invention provides a Wnt3-Fzd1 chimeric protein, i.e., a Wnt3-Fzd1 chimera.

The invention also relates to a Wnt-Fzd chimera, especially the Wnt3-Fzd1 chimeric protein, that has a cytoplasmic tail involved in signal transduction. In one embodiment, the chimera has a modified cytoplasmic tail. In another embodiment, the chimera lacks a cytoplasmic tail.

The present invention also relates to a Wnt-Fzd chimera, and particularly to the Wnt3-Fzd1 chimeric protein, that has a modified CRD domain of an Fzd1 portion. These modifications include mutation of the tyrosine residue involved in Wnt signaling (depicted in FIG. 2), and deletion of a portion of the CRD domain, e.g., a 29 amino acid residue deletion.

The invention also provides a nucleic acid encoding the chimeric Wnt-Fzd proteins, especially the Wnt3-Fzd1 chimeras, described above.

The invention further includes expression vectors comprising the foregoing nucleic acids, host cells transfected with such expression vectors, and methods for making the chimeric proteins by culturing the host cells.

The invention also provides a system for studying Wnt signaling, comprising such host cells, as well as a method for studying Wnt signaling, which method comprises studying the system of the invention.

These and other aspects of the invention will be better understood by reference to the drawings, detailed description, and example section.

### BRIEF DESCRIPTION OF THE DRAWINGS

FIG. 1A is an alignment of portions of the sequences of human Fzd proteins 1-10 (HFZ1-HFZ10; SEQ ID NOS: 11-20, respectively) and murine Fzd proteins 1, 3, 4, and 6-9 (MFZ1, MFZ3, MFZ4, MFZ6-9; SEQ ID NOS: 21-27, respectively, respectively).

FIG. 1B shows a portion of the human Fzd1 sequence (SEQ ID NO: 11) with the mutated position (YN to AA) is underlined, and the deleted CRD is shown with arrows.

FIG. 2 provides the amino acid sequences of Fzd CRDs between the second and third cysteine residues for Fzd1-Fzd10 (portions of SEQ ID NOS: 11-20, respectively). The CRD domain YN residues are indicated in bold and with underlining.

FIG. 3 is a graph demonstrating that the CRD domain YN residues (see FIG. 2) are critical for Wnt signaling of the Wnt3-Fzd1 chimeras.

FIG. 4 is a graph showing that the Wnt3-Fzd1 chimera is efficient in activating Wnt signaling in U2OS cells.

FIG. 5 is a graph showing that the cytoplasmic tail of Wnt3-Fzd1 chimeras is critical for canonical Wnt signaling.

FIG. 6 is a graph showing that the Wnt3-Fzd1 chimera activates canonical Wnt signaling in cis, but not on neighboring cells.

FIG. 7 is a graph showing that the Wnt3-Fzd1 CRD domain is critical for Wnt signaling.

FIG. 8 is an alignment of the amino acid sequences of SFRPs 1-5 (SEQ ID NOS: 3, 5, 6, 7, and 4, respectively).

FIG. 9 is a graph showing the Dkk1 completely abolishes Wnt signaling induced by the Wnt3-Fzd1 chimera in U2OS cells.

FIG. 10 is a graph showing Wnt3-Fzd1 chimera is a potent activator of C3H10T1/2 cell differentiation into osteoblasts in which the C3H10T1/2 cells were grown in growth medium.

FIG. 11 is a graph showing Wnt3-Fzd1 chimera is a potent activator of C3H10T1/2 cell differentiation into osteoblasts in which the C3H10T1/2 cells were grown in differentiation medium.

### DETAILED DESCRIPTION

A chimera of Wnt3-Fzd1 has been developed to address the role of Wnt signaling and to understand the critical regions within the Wnt and Fzd molecules required for optimal Wnt signaling. The chimera is very effective in driving the canonical Wnt signaling. A deletion of the Fzd cytoplasmic tail or its mutation drastically reduces the Wnt signaling, reinforcing the role of the Fzd receptor cytoplasmic region in Wnt signaling.

A small deletion of the CRD domain (29 amino acids) completely abolishes the Wnt signaling ability of Wnt3-Fzd1 chimera and the results indicate the importance of the Fzd CRD domain in Wnt signaling. The change of two well-conserved tyrosines and asparagines into two alanine residues have also resulted in total loss of Wnt signaling by the chimera protein. The results clearly demonstrate the critical role of these two amino acids in the Wnt signaling function of the Fzd receptor. The above study reinforces the observation of the importance of the tyrosine residue in the function of SFRPs and also extends the critical role of tyrosine residue in the Wnt signaling function of Fzd receptors.

The Example section below validates the Wnt-Fzd chimera as a suitable model to study Wnt signaling.

Accordingly, the present invention provides for a Wnt-Fzd chimera, specifically a Wnt3-Fzd1 chimera. In one embodiment of the invention, the chimeras or nucleic acids encoding them are used for developing treatments of diseases that involve Wnt signaling. The chimeras may be introduced into, for example, but not limited to, organisms, tissues, or cells. The chimeras can be constitutively active or, when the mutations disclosed below are introduced, may have any range of Wnt signaling activities. The Wnt signaling activity of the chimera can be altered using single or multiple site mutations or deletions. Furthermore, Wnt signaling activity can be altered using frame shift mutations.

Wnt signaling is initiated by the binding of Wnt to a membrane receptor complex composed of the Fzd receptor and low-density lipoprotein receptor-related protein (LRP), leading to the activation of the canonical, Wnt/β-catenin, pathway. The activation of Wnt signaling can be measured either by the increase in the cytoplasmic accumulation of β-catenin or the activation of T-cell factor/lymphoid enhancer factor (TCF/LEF)-reporter genes (Wodarz et al. (1998) Annu. Rev. Cell Dev. Biol. 14:59-88; Miller (2002) Genome Biol. 3: reviews3001.1-3001.15). In such assays, SFRP-1 is shown to decrease the Wnt-mediated accumulation of cytoplasmic β-catenin and inhibit the activation of the TCF-reporter. Microinjection of mRNA into Xenopus embryos is generally used to validate Wnt signaling, and in such a system, Wnt1-induced axis duplication is inhibited by SFRPs (Lin et al. (1997) Proc. Natl. Acad. Sci. USA 94:11196-11200). Biochemical studies utilizing the co-immunoprecipitation or ELISA methods are also used to identify the interaction of Wnt and SFRPs; however; the results of such physical interaction studies do not correlate with in vivo functional studies using Xenopus embryo axis duplication (Lin et al. (1997) Proc. Natl. Acad. Sci. USA 94:11196-11200).

Structure-function studies using bovine SFRP-3 mutants have revealed that the complete removal of the CRD abolishes the Wnt1/SFRP-3 interaction in vitro and the inhibition of the Wnt1-mediated axis duplication in Xenopus embryos (Lin et al. (1997) Proc. Natl. Acad. Sci. USA 94:11196-11200). In contrast, removal of the carboxyl-terminal portion of the molecule preserves both the Wnt-SFRP-3 interaction and reduced functional inhibition of axis duplication. However, studies utilizing human SFRP-1 and Wg (wingless, a Drosophila Wnt) have shown that the SFRP mutants lacking the CRD retained the ability to bind to Wg, and the deletion of the carboxyl terminal resulted in the reduction or loss of Wg binding. These studies have concluded that the CRD might confer a component of the binding capacity, but the carboxyl-terminal region of the SFRP-1 is primarily responsible for its ability to bind Wg (Uren et al. (2000) J. Biol. Chem. 275:4374-4382). Although the above methods provided the insight into the potential mechanism of the Wnt antagonism of SFRP-1, the studies failed to identify the critical regions that are essential to the biological function of SFRPs.

In the Example section below, an optimized TCF-Luciferase reporter-based assay was used for measuring Wnt signaling. A luciferase-based reporter plasmid containing 16 copies of the TCF-element upstream of a tk promoter was developed (Bhat et al. (2004) Protein Expr. Purif. 37:327-335). Several cell lines were analyzed for the optimal Wnt response, and the U2OS cells reproducibly showed a good Wnt response with a nearly 30-fold activation when co-transfected with a Wnt3 expression plasmid. The amount of Wnt and hOB SFRP-1 transfected were optimized to obtain nearly 90% inhibition with SFRP-1. This optimized transfection method allowed characterization of the SFRP-1 mutants and to identify the critical regions that are required for Wnt antagonist function. Using this assay system, it was determined that human SFRP-3 is less efficient in inhibiting Wnt3 compared to SFRP-1, which could be due to the differences in the CRD sequences. A change of the sequences in the 2^{nd} loop of-SFRP-1 compared to those of SFRP-3 have identified that the amino acids between 73-86 play an important role in the Wnt antagonist function of SFRP-1. In particular, the change from KKMVL (SEQ ID NO: 28) to NMTKM (SEQ ID NO: 29) leads to a substantial loss of the antagonist activity. The results correlate very well with studies of alanine scanning mutants of mouse SFRP-3 CRD and its subsequent binding to an XWnt8-AP chimera. Mutations around NMTKM (SEQ ID NO: 29) lead to either reduced or total loss of the binding of the SFRP-3 mutants to XWnt-AP (Dann et al. (2001) Nature 412:86-90). Similarly, a change from LLEHE (SEQ ID NO: 30) to HLHHS (SEQ ID NO: 31) (as in SFRP-3) affected the Wnt antagonist function of SFRP-1. In SFRP-3, the H-HHS residues are exposed residues based on fractional solvent solubility studies, and it is possible that subtle changes in the amino acids of SFRPs may alter its secondary and tertiary structures, affecting the Wnt antagonist function of SFRP.

It is intriguing to note the critical role of tyrosine in SFRP-1 (amino acid 73) and the corresponding amino acid in Fzds and their Wnt antagonist function. This tyrosine is conserved in closely related SFRPs like SFRP-1 (human sequence set forth in SEQ ID NO: 3), SFRP-2 (human sequence set forth in SEQ ID NO: 4) and SFRP-5 (human sequence set forth in SEQ ID NO: 5) and is replaced by tryptophan in SFRP-3 (human sequence set forth in SEQ ID NO: 6) and SFRP-4 (human sequence set forth in SEQ ID NO: 7). The tyrosine is also conserved in all of the frizzled receptors. The change of tyrosine to tryptophan in SFRP-1 did not affect its Wnt antagonist function, whereas a change to phenylalanine did result in about a 20% loss of Wnt antagonist function. A more drastic effect on Wnt antagonist function is seen when the aromatic amino acid, tyrosine, is changed into a neutral or polar amino acid such as alanine, serine, and aspartic or asparagine. In crystal structure studies with the mouse SFRP-3 CRD and mFzd8 CRD, the tryptophan/tyrosine residue is buried within the CRD structure. In many proteins, tyrosine residues are generally involved in H bonding, either with other amino acid side chains or with water molecules. The change of the aromatic amino acid tyrosine into a neutral or polar amino acid may disrupt such bonding, altering the folding of the molecule and resulting in the loss of Wnt antagonist function of SFRPs. Preliminary studies with the frizzled receptor have shown that the tyrosine residue in the 2^{nd} loop indeed is critical for the activation of canonical signaling by Wnt ligand. The change of tryptophan to tyrosine in SFRP-3 results in gain of Wnt antagonist activity, suggesting that the tyrosine residue is the favored amino acid residue for optimal Wnt antagonist function of SFRPs.

In defining the terms of the present invention, the term “bone formation” is the process of bone synthesis and mineralization. The term “bone-forming activity” is defined as performing the process of bone formation. The term “osteogenesis” is synonymous with the term bone formation, defined above. The term “bone growth” is the process of skeletal expansion. This process occurs by one of two ways: (1) intramembraneous bone formation arises directly from mesenchymal or bone marrow cells; (2) longitudinal or endochondral bone formation arises where bone forms from cartilage. The term “bone density” refers to the amount of bone tissue per a certain volume within bone. Low bone density is often associated with bone disorders, such as osteoporosis. The term “secreted frizzled related proteins” or “SFRP” is a secreted receptor of the Wnt signaling pathway and exhibits a number of characteristics that make it a useful tool for studying cell growth and differentiation. “SFRP activity” refers to any of the biological activities of the native SFRP protein molecule, including, but not limited to, antagonism of the Wnt signaling pathway. The terms “secreted apoptosis related protein” and “SARP” are synonymous with the terms secreted frizzled related protein and SFRP, defined above. The terms hOB SFRP, FRP-1, FrzA and SARP-2 are synonymous with the term SFRP-1.

The present invention encompasses methods using any Wnt-Fzd proteins and mutated forms of the proteins found to be valuable for use in these methods. Also encompassed are the test compounds discovered through use of these methods.

As used herein, a “Wnt-Fzd chimeric protein,” also termed a “Wnt-Fzd chimera,” is a construct in which a Wnt protein is joined at the N-terminus of a Frizzled (Fzd/Fzd) receptor protein. Thus, the chimera comprises a Wnt portion and a Fzd portion. These two portions are joined in such a way that the Wnt portion is capable of inducing signal transduction by the Fzd portion. However, Wnt-Fzd chimeras of the invention also include modified forms which alter or inhibit signal transduction. These modified chimeric proteins include alterations of the cytoplasmic tail and/or the CRD domain, both found in the Fzd portion of the chimera.

The term “Wnt” refers to the Wnt family of glycosylated proteins discussed in the Background. It specifically includes the proteins depicted in SEQ ID NOS: 1 and 2. In specific embodiment, Wnt is Wnt3.

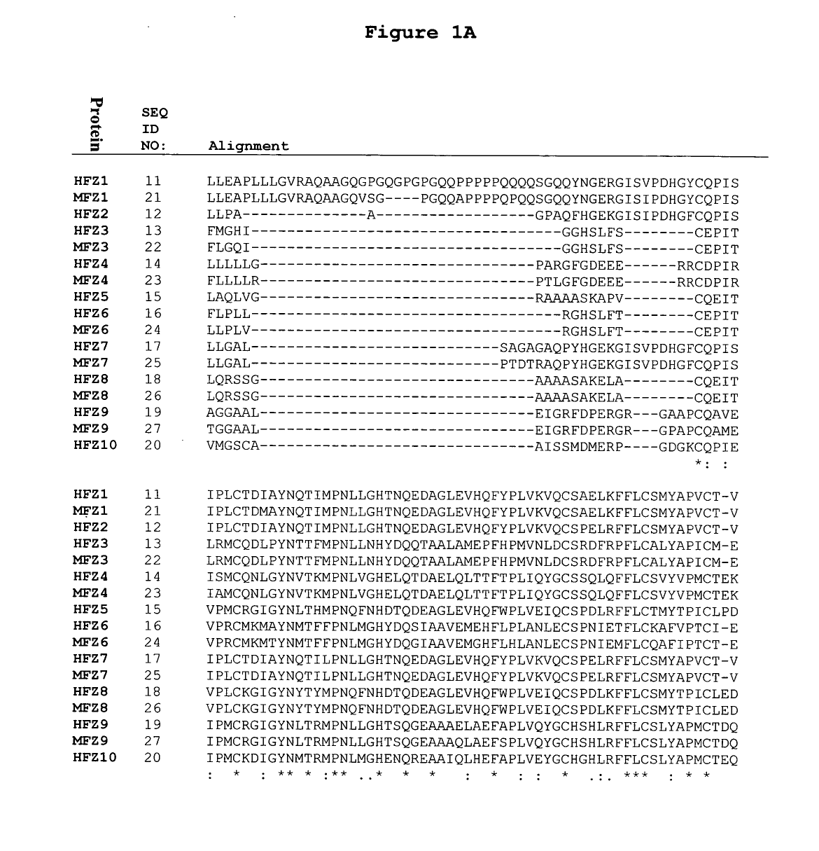

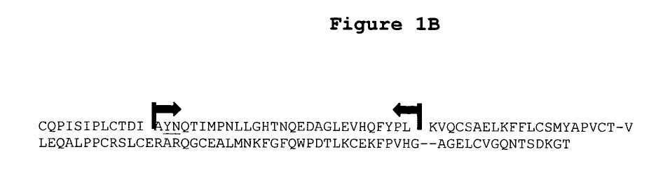

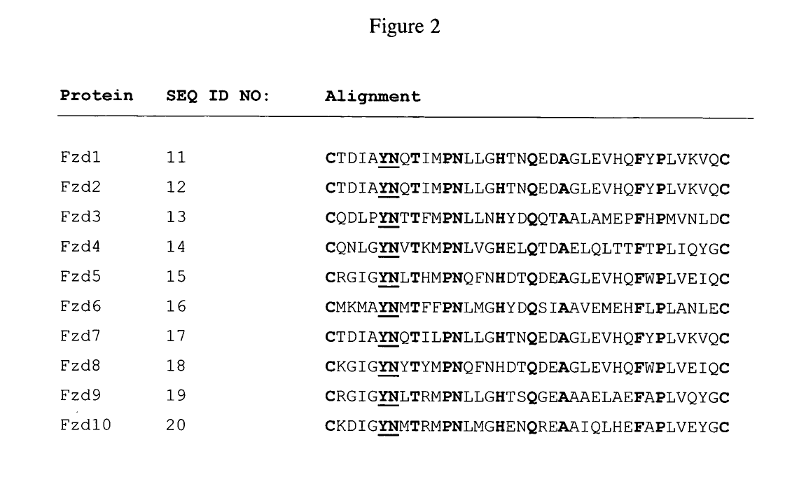

The term “Fzd” refers to the Frizzled family of proteins discussed in the Background. It specifically includes the proteins depicted in FIG. 1. In a specific embodiment, Fzd is Fzd1.

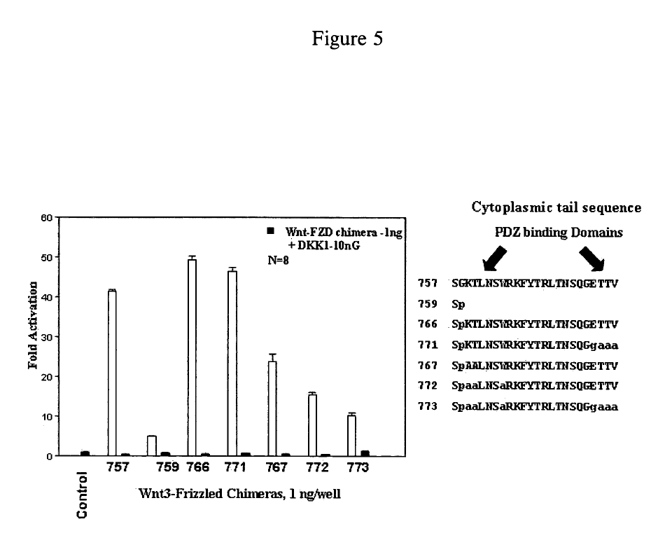

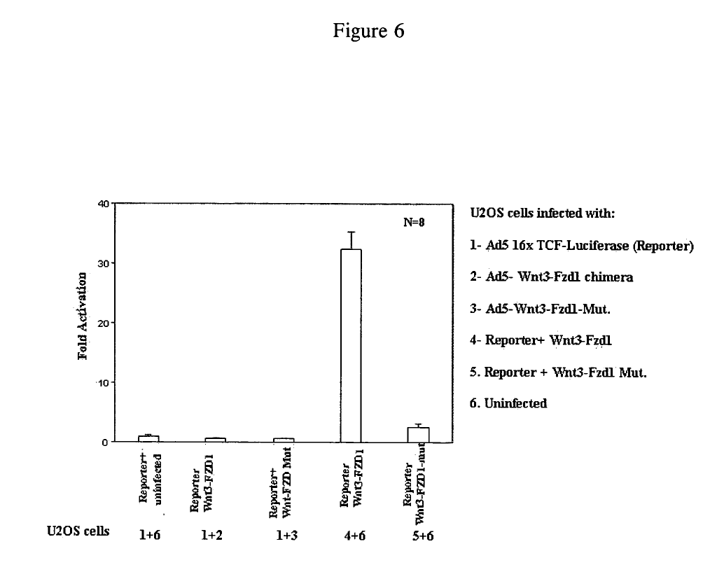

A “modified cytoplasmic tail” means a Wnt-Fzd chimera in which the cytoplasmic tail of the Fzd portion has been altered (for example by altering the amino acid sequence) or deleted, especially in the PZD binding domains, thereby changing the signal transduction properties of the chimera. The Example section and FIG. 5 provide specific embodiments of a Wnt-Fzd chimera in which the Fzd is Fzd1, and the cytoplasmic tail is altered or deleted.

The term “cysteine-rich domain” or “CRD” refers to a protein domain which has 10 conserved cysteines in its primary structure (amino acid sequence) that form five disulfide bridges. This domain is mainly α-helical in structure.

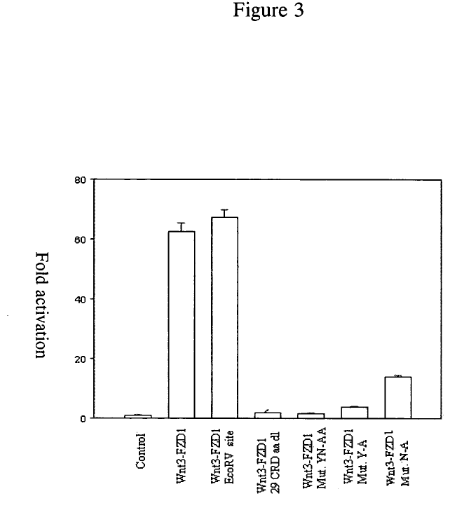

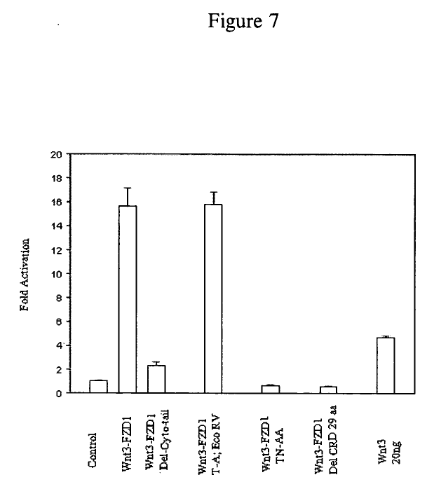

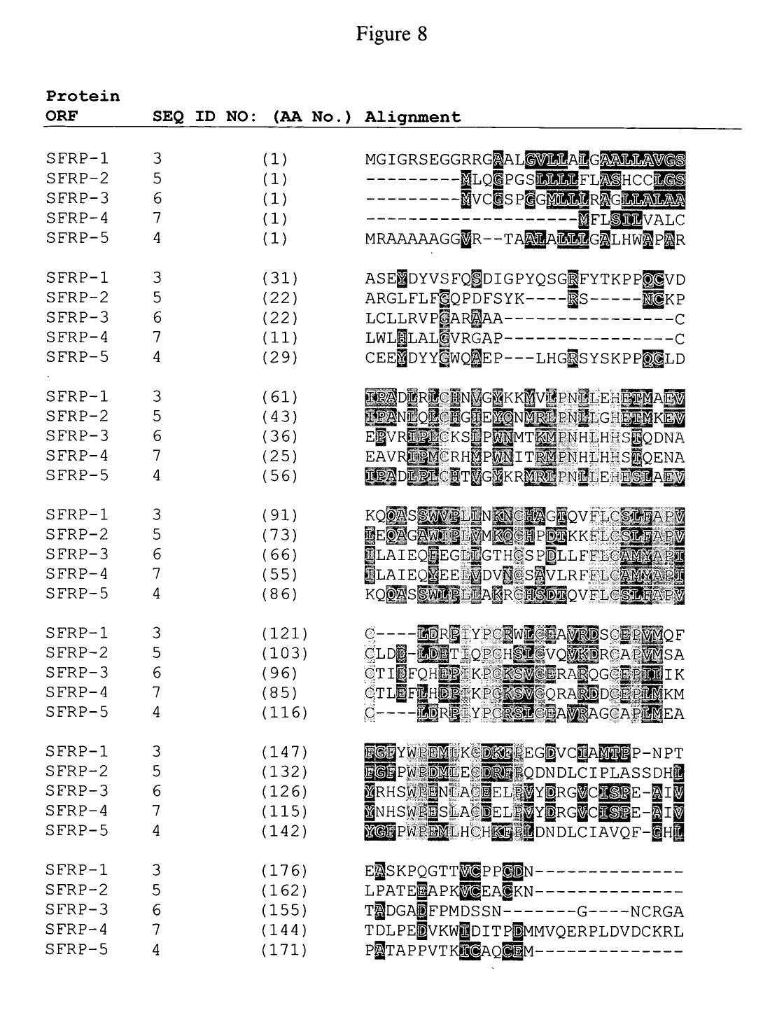

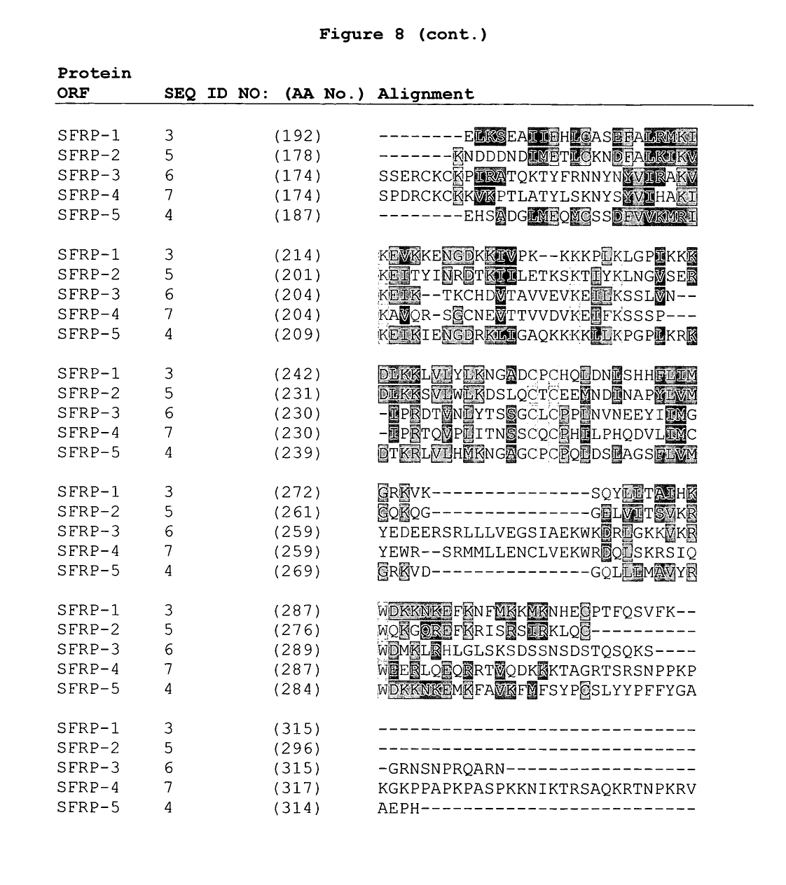

A “modified CRD domain” refers to a Wnt-Fzd chimera in which the CRD domain of the Fzd portion of the chimera is altered to change the amino acid sequence or delete amino acid residues, e.g., as shown in the Example section and FIGS. 3 and 7 for Fzd1.

The Wnt-signaling pathway may propagate a signal through several different mechanisms (Miller (2001) Genome Biol. 3: reviews3001.1-3001.15). Without being bound to any theory (an understanding of the mechanism is not necessary to practice the present invention, and the present invention is not limited to any particular mechanism), these include the Wnt/β-catenin, Wnt/Ca²⁺, and Wnt/polarity pathways. In the Wnt/β-catenin (also known as the canonical Wnt-signaling pathway) pathway, Wnt binds the Frizzled receptor which propagates a signal to inhibit the phosphorylation of β-catenin by glycogen synthase kinase-3 (GSK-3). Phosphorylated β-catenin would be ubiquitinated and degraded; however, by blocking GSK-3, β-catenin accumulates within the cell nucleus and activates T-cell factor/lymphoid enhancer factor (TCF/LEF) transcription factors, upregulating target genes. In the Wnt/Ca²⁺ pathway, the non-canonical Wnt pathway, Wnt activation of Fz results in a G-protein coupled response and the release of calcium into the cytoplasm from the endoplasmic reticulum. The increased calcium concentration activates protein kinase C (PKC) and calcium/calmodulin-regulated kinase II (CamKII). The Wnt/Ca²⁺ pathway antagonizes the Wnt/β-catenin pathway. Finally, the Wnt/polarity pathway regulates cytoskeletal organization and cellular axis determination.

“Wnt activity” refers to any of the biological activities of the native Wnt protein molecule and can be measured using a variety of methods, including measuring a change in one of the Wnt signaling pathways. One method involves the use of a TCF-luciferase assay. This assay uses a target reporter gene (luciferase) under the control of a TCF responsive element linked to a minimal promoter as described in the Example section below. The TCF-luciferase assay can be used with any cell type. The effect of Wnt signaling on bone-related genes may be determined using the TCF-luciferase assay with bone cell types. Successful Wnt signaling produces a luciferase-mediated signal whereas a reduction in this signal indicates inhibition of the Wnt signaling pathway. Wnt activity may also be measured using transgenic animals, as described below. The effect of Wnt signaling on bone may be determined by measuring bone-related parameters. Further measurements may be performed using any of the pathway mechanisms described above or others that may be subsequently discovered. The measurement of Wnt activity as described is not limited to these examples.

“Incubating” a “sample” refers to, especially when used in terms of measuring Wnt activity, any method of having items, such that it may be determined if they interact with one another or are associated with a particular response. For example, “incubating a first sample comprising” a molecule “and the test compound” means any method of bring a molecule and test compound together. The molecule may be purified and used in a cell-free assay with purified test compound. Alternatively, the molecule may be in a cell lysate assay with purified test compound. Furthermore, the molecule may be encoded within DNA inserted within a cell that is exposed to the test compound. The molecule and test compound may be determined to bind one another in a binding experiment or assay. The molecule and test compound may be determined to be associated with a response such as Wnt activity. It is well appreciated within the art that many different systems may be used to test the interaction of two or more molecules, and the above examples by no means limit the invention.

Particular domains and specific amino acids of SFRPs are important in the Wnt-signaling pathway. SFRPs inhibit the interaction of Wnt with Fz. It has been found that mutation or deletion of specific domains reduces the inhibition of SFRP for Wnt signaling, thus providing disinhibition, and allowing an increase in Wnt-signaling. An increase in Wnt-signaling promotes increased rate of bone formation, a decrease in osteoblast and osteoclast apoptosis, and an increase in osteoblast differentiation. The important domains include the CRD, netrin, hyaluronan domains, and the carboxy-terminal region.

The terms “proteins,” “peptides” and “polypeptides” are used interchangeably and are intended to include purified and recombinantly produced molecules containing amino acids linearly coupled through peptide bonds. The amino acids can be in the L or D form so long as the biological activity of the polypeptide is maintained. The proteins may also include proteins that are post-translationally modified by reactions that include, but are not limited to, glycosylation, acetylation, or phosphorylation. Such polypeptides also include analogs, alleles, and allelic variants that can contain amino acid derivatives or non-amino acid moieties that do not affect the biological or functional activity of the protein as compared to wild-type or naturally occurring protein. The term “amino acid” refers both to the naturally occurring amino acids and their derivatives, such as TyrMe and PheCl, as well as other moieties characterized by the presence of both an available carboxyl group and an amine group. Non-amino acid moieties that can be contained in such polypeptides include, for example, amino acid mimicking structures. Mimicking structures are those structures that exhibit substantially the same spatial arrangement of functional groups as amino acids but do not necessarily have both the amino and carboxyl groups characteristic of amino acids.

“Muteins” are protein or polypeptide “mutants” that have minor changes, i.e. “mutations,” in amino acid sequence caused, for example, by site-specific mutagenesis or other manipulations, by errors in transcription or translation, or which are prepared synthetically by rational design. These minor alterations result in amino acid sequences which may alter a biological activity or other characteristics of the protein or polypeptide compared to wild-type or naturally occurring polypeptide or protein.

The phrases “corresponding to” and “corresponds to,” when applied to domains or amino acids within a protein, means the comparable domain or amino acid, respectively, within a protein using a Wnt or SFRP as a reference. For example, SFRPs from separate animal species or individual SFRPs of one animal species may not have identical sequences. However, upon sequence alignment, it is appreciated by those who possess ordinary skill in the art that, even though aligned amino acids may have different numbering within their respective sequences, the domains or amino acids that align (even if not identical) are domains or amino acids that correspond to one another, respectively.

“Isolated,” when referring to a nucleic acid molecule, means separated from other cellular components.

“Purified” when referring to a protein or polypeptide, is distinguishable from native or naturally occurring proteins or polypeptides because they exist in a purified state. These “purified” proteins or polypeptides, or any of the intended variations as described herein, shall mean that the compound or molecule is substantially free of contaminants normally associated with the compound in its native or natural environment. The terms “substantially pure” and “isolated” are not intended to exclude mixtures of polynucleotides or polypeptides with substances that are not associated with the polynucleotides or polypeptides in nature.

A “purification tag” is any molecular moiety added to a peptide or protein to aid in the purification process. Purification tags well known in the art include, but are not limited to, antibody recognition tags (affinity tags, e.g. myc epitope tag), histidine (His) tags, streptavidin binding peptide (SBP) tags, maltose binding protein (MBP) tags, and glutathione S-transferase (GST) tags.

“Native” polypeptides, proteins, or nucleic acid molecules refer to those recovered from a source occurring in nature or “wild-type.”

The term “small molecule” refers to a compound that has a molecular weight of less than about 2000 Daltons, less than about 1000 Daltons, or less than about 500 Daltons. Small molecules, without limitation, may be, for example, nucleic acids, peptides, polypeptides, peptide nucleic acids, peptidomimetics, carbohydrates, lipids, or other organic (carbon containing) or inorganic molecules and may be synthetic or naturally occurring or optionally derivatized. Such small molecules may be a therapeutically deliverable substance or may be further derivatized to facilitate delivery or targeting.

The term “nucleic acid” means single and double-stranded DNA, cDNA, genome-derived DNA, and RNA, as well as the positive and negative strand of the nucleic acid that are complements of each other, including anti-sense RNA. A “nucleic acid molecule” is a term used interchangeably with “polynucleotide” and each refers to a polymeric form of nucleotides of any length, either ribonucleotides or deoxyribonucleotides, or analogs thereof. It also includes known types of modifications, for example, labels which are known in the art (e.g., Sambrook et al., (1989) infra.), methylation, “caps,” substitution of one or more of the naturally occurring nucleotides with an analog, internucleotide modifications such as, for example, those with uncharged linkages (e.g., methyl carbamate, etc.), those containing pendant moieties, such as for example, proteins (including, e.g., nuclease, toxins, antibodies, signal peptides, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide. The polynucleotide can be chemically or biochemically modified or contain non-natural or derivatized nucleotide bases. The nucleotides may be complementary to the mRNA encoding the polypeptides. These complementary nucleotides include, but are not limited to, nucleotides capable of forming triple helices and antisense nucleotides. Recombinant polynucleotides comprising sequences otherwise not naturally occurring are also provided by this invention, as are alterations of wild-type polypeptide sequences, including but not limited to, those due to deletion, insertion, substitution of one or more nucleotides or by fusion to other polynucleotide sequences.

A polynucleotide is said to “encode” a polypeptide if, in its native state or when manipulated by methods well-known to those skilled in the art, it can be transcribed and/or translated to produce a polypeptide or mature protein. Thus, the term polynucleotide shall include, in addition to coding sequences, processing sequences and other sequences that do not code for amino acids of the mature protein. The anti-sense strand of such a polynucleotide is also said to encode the sequence.

The term “recombinant” polynucleotide or DNA refers to a polynucleotide that is made by the combination of two otherwise separated segments of sequence accomplished by the artificial manipulation of isolated segments of DNA by genetic engineering techniques or by chemical synthesis. In so doing, one may join together DNA segments of desired functions to generate a desired combination of functions.

An “analog” of a DNA, RNA or polynucleotide refers to a macromolecule resembling naturally occurring polynucleotides in form and/or function (particularly in the ability to engage in sequence-specific hydrogen bonding to base pairs on a complementary polynucleotide sequence) but which differs from DNA or RNA in, for example, the possession of an unusual or non-natural base or an altered backbone. See, for example, Uhlmann et al. (1990) Chem. Rev. 90:543-584.

“Hybridization” refers to hybridization reactions that can be performed under conditions of different “stringency”. Conditions that increase the stringency of a hybridization reaction are widely known and published in the art: see, for example, Sambrook et al., infra. Examples of relevant conditions include (in order of increasing stringency): incubation temperatures of 25° C., 37° C., 50° C., and 68° C.; buffer concentrations of 10×SSC, 6×SSC, 1×SSC, 0.1×SSC (where SSC is 0.15 M NaCl and 15 mM citrate buffer) and their equivalent using other buffer systems; formamide concentrations of 0%, 25%, 50%, and 75%, incubation times from 5 minutes to 24 hours and washes of increasing duration, increasing frequency, or decreasing buffer concentrations.

“Tm” is the temperature in degrees Centigrade at which 50% of a polynucleotide duplex made of complementary strands hydrogen bonded in an antiparallel direction by Watson-Crick base paring dissociates into single strands under the conditions of the experiment. Tₘ may be predicted according to standard formulas, for example:
Tₘ=81.5+16.6log[Na⁺]+0.41(% G/C)−0.61(% F)−600/L
where [Na⁺] is the cation concentration (usually sodium ion) in mol/L; (% G/C) is the number of G and C residues as a percentage of total residues in the duplex; (% F) is the percent formamide in solution (wt/vol); and L is the number of nucleotides in each strand of the duplex.

A “stable duplex” of polynucleotides, or a “stable complex” formed between any two or more components in a biochemical reaction, refers to a duplex or complex that is sufficiently long lasting to persist between the formation of the duplex or complex, and its subsequent detection. The duplex or complex must be able to withstand whatever conditions exist or are introduced between the moment of formation and the moment of detection, these conditions being a function of the assay or reaction which is being performed. Intervening conditions which may optionally be present and which may dissociate a duplex or complex include washing, heating, adding additional solutes or solvents to the reaction mixture (such as denaturants), and competing with additional reacting species. Stable duplexes or complexes may be irreversible or reversible, but must meet the other requirements of this definition. Thus, a transient complex may form in a reaction mixture, but it does not constitute a stable complex if it dissociates spontaneously or as a result of a newly imposed condition or manipulation introduced before detection.

When stable duplexes form in an antiparallel configuration between two single-stranded polynucleotides, particularly under conditions of high stringency, the strands are essentially “complementary.” A double-stranded polynucleotide can be “complementary” to another polynucleotide, if a stable duplex can form between one of the strands of the first polynucleotide and the second. A complementary sequence predicted from the sequence of single stranded polynucleotide is the optimum sequence of standard nucleotides expected to form hydrogen bonding with the single-stranded polynucleotide according to generally accepted base-pairing rules.

A “sense” strand and an “antisense” strand when used in the same context refer to single-stranded polynucleotides which are complementary to each other. They may be opposing strands of a double-stranded polynucleotide, or one strand may be predicted from the other according to generally accepted base-pairing rules. Unless otherwise specified or implied, the assignment of one or the other strand as “sense” or “antisense” is arbitrary.

A linear sequence of nucleotides is “identical” to another linear sequence if the order of nucleotides in each sequence is the same, and occurs without substitution, deletion, or material substitution. It is understood that purine and pyrimidine nitrogenous bases with similar structures can be functionally equivalent in terms of Watson-Crick base-pairing; and the inter-substitution of like nitrogenous bases, particularly uracil and thymine, or the modification of nitrogenous bases, such as by methylation, does not constitute a material substitution so long as the substitution does not alter hydrogen bonding between the bases. An RNA and a DNA polynucleotide have identical sequences when the sequence for the RNA reflects the order of nitrogenous bases in the polyribonucleotide, the sequence for the DNA reflects the order of nitrogenous bases in the polydeoxyribonucleotide, and the two sequences satisfy the other requirements of this definition. Where at least one of the sequences is a degenerate oligonucleotide comprising an ambiguous residue, the two sequences are identical if at least one of the alternative forms of the degenerate oligonucleotide is identical to the sequence with which it is being compared. For example, AYAAA is identical to ATAAA, if AYAAA is a mixture of ATAAA and ACAAA and AYAAA is being compared to ATAAA.

When comparison is made between polynucleotides, it is implicitly understood that complementary strands are easily generated, and the sense or antisense strand is selected or predicted that maximizes the degree of identity between the polynucleotides being compared. For example, where one or both of the polynucleotides being compared is double-stranded, the sequences are identical if one strand of the first polynucleotide is identical with one strand of the second polynucleotide. Similarly, when a polynucleotide probe is described as identical to its target, it is understood that it is the complementary strand of the target that participates in the hybridization reaction between the probe and the target.

A linear sequence of nucleotides is “essentially identical” or the “equivalent” to another linear sequence if both sequences are capable of hybridizing to form duplexes with the same complementary polynucleotide. It should be understood, although not always explicitly stated, that when Applicants refer to a specific nucleic acid molecule, its equivalents are also intended. Sequences that hybridize under conditions of greater stringency are one embodiment. It is understood that hybridization reactions can accommodate insertions, deletions, and substitutions in the nucleotide sequence. Thus, linear sequences of nucleotides can be essentially identical even if some of the nucleotide residues do not precisely align. Sequences that align more closely to the invention disclosed herein are another embodiment. Generally, a polynucleotide region of about 25 residues is essentially identical to another region if the sequences are at least about 85% identical, at least about 90% identical, at least about 95% identical, or 100% identical. A polynucleotide region of 40 residues or more will be essentially identical to another region, after alignment of homologous portions, if the sequences are at least about 85% identical, at least about 90% identical at least 95% identical, or 100% identical.

The phrases “corresponding to” and “corresponds to,” when applied to nucleic acids, means the comparable base within a nucleic acid molecule using a Wnt or SFRP as a reference. For example, SFRPs from separate animal species or individual SFRPs of one animal species may not have identical sequences. However, upon sequence alignment, it is appreciated by those who possess ordinary skill in the art that, even though aligned bases may have different numbering within their respective sequences, the bases that align are bases that correspond to one another.

In determining whether polynucleotide sequences are essentially identical, a sequence that preserves the functionality of the polynucleotide with which it is being compared is one embodiment. Functionality can be determined by different parameters. For example, if the polynucleotide is to be used in reactions that involve hybridizing with another polynucleotide, then preferred sequences are those which hybridize to the same target under similar conditions. In general, the Tₘ of a DNA duplex decreases by about 10° C. for every 1% decrease in sequence identity for duplexes of 200 or more residues; or by about 50° C. for duplexes of less than 40 residues, depending on the position of the mismatched residues (see, e.g. Meinkoth and Wahl (1984) Anal. Biochem. 138:267-284). Essentially identical or equivalent sequences of about 100 residues will generally form a stable duplex with each other's respective complementary sequence at about 20° C. less than Tₘ, at about 15° C. less, at about 10° C. less, at about 5° C. less, at about Tₘ. In another example, if the polypeptide encoded by the polynucleotide is an important part of its functionality, then preferred sequences are those which encode identical or essentially identical polypeptides. Thus, nucleotide differences which cause a conservative amino acid substitution are one embodiment; nucleotide differences which cause non-conservative amino acid substitutions are another embodiment; nucleotide differences which do not alter the amino acid sequence are an embodiment while identical nucleotides are yet another embodiment. Insertions or deletions in the polynucleotide that result in insertions or deletions in the polypeptide are embodiments whereas those that result in the down-stream coding regions being rendered out of phase are another embodiment; polynucleotide sequences comprising no insertions or deletions are another embodiment. The relative importance of hybridization properties and the encoded polypeptide sequence of a polynucleotide depend on the application of the invention.

A polynucleotide has the same characteristics or is the equivalent of another polynucleotide if both are capable of forming a stable duplex with a particular third polynucleotide under similar conditions of maximal stringency. Preferably, in addition to similar hybridization properties, the polynucleotides also encode essentially identical polypeptides.

“Conserved” residues of a polynucleotide sequence are those residues that occur unaltered in the same position of two or more related sequences being compared. Residues that are relatively conserved are those that are conserved amongst more related sequences than residues appearing elsewhere in the sequences.

As used herein, a “degenerate” oligonucleotide sequence is a designed sequence derived from at least two related originating polynucleotide sequences as follows: the residues that are conserved in the originating sequences are preserved in the degenerate sequence, while residues that are not conserved in the originating sequences may be provided as several alternatives in the degenerate sequence. For example, the degenerate sequence AYASA may be assigned from originating sequences ATACA and ACAGA, where Y is C or T and S is C or G. Y and S are examples of “ambiguous” residues. A degenerate segment is a segment of a polynucleotide containing a degenerate sequence.

It is understood that a synthetic oligonucleotide comprising a degenerate sequence can be a mixture of closely related oligonucleotides sharing an identical sequence, except at the ambiguous positions. Such a mixture of all possible combinations of nucleotides may be produced by synthetic methods. Each of the oligonucleotides in the mixture is referred to as an “alternative form.”

A polynucleotide “fragment” or “insert” as used herein generally represents a sub-region of the full-length form, but the entire full-length polynucleotide may also be included.

Different polynucleotides “relate” to each other if one is ultimately derived from another. For example, messenger RNA relates to the gene from which it is transcribed. cDNA relates to the RNA from which it has been produced, such as by a reverse transcription reaction, or by chemical synthesis of a DNA based upon knowledge of the RNA sequence or the coding sequence of genomic DNA. cDNA also relates to the coding sequence of the gene that encodes the RNA. Polynucleotides also “relate” to each other if they serve a similar function, such as encoding a related polypeptide in different species, strains or variants that are being compared.

The term “upstream” refers to nucleic acid base(s) or base pair(s) that are 5′ to the reference nucleic acid base(s) within a nucleic acid molecule, the 5′ determined using the sense strand, or the strand derived from the sense strand, if the nucleic acid molecule is double stranded.

A “probe” when used in the context of polynucleotide manipulation refers to an oligonucleotide that is provided as a reagent to detect a target potentially present in a sample of interest by hybridizing with the target. Usually, a probe will comprise a label or a means by which a label can be attached, either before or subsequent to the hybridization reaction. Suitable labels include, but are not limited to, radioisotopes, fluorochromes, chemiluminescent compounds, dyes, and proteins, including enzymes.

A “primer” is an oligonucleotide, generally with a free 3′-OH group, that binds to a target potentially present in a sample of interest by hybridizing with the target, and thereafter promotes polymerization of a polynucleotide complementary to the target.

Processes of producing replicate copies of the same polynucleotide, such as PCR or gene cloning, are collectively referred to herein as “amplification” or “replication”. For example, single or double-stranded DNA may be replicated to form another DNA with the same sequence. RNA may be replicated, for example, by an RNA-directed RNA polymerase, or by reverse-transcribing the DNA and then performing a PCR. In the latter case, the amplified copy of the RNA is a DNA with the identical sequence.

Elements within a gene include, but are not limited to, promoter regions, enhancer regions, repressor binding regions, transcription initiation sites, ribosome binding sites, translation initiation sites, protein encoding regions, introns and exons, and termination sites for transcription and translation. An “antisense” copy of a particular polynucleotide refers to a complementary sequence that is capable of hydrogen bonding to the polynucleotide and can therefore, be capable of modulating expression of the polynucleotide. These are DNA, RNA or analogs thereof, including analogs having altered backbones, as described above. The polynucleotide to which the antisense copy binds may be in single-stranded form or in double-stranded form.

As used herein, the term “operatively linked” means that the DNA molecule is positioned relative to the necessary regulation sequences, e.g., a promoter or enhancer, such that the promoter will direct transcription of RNA off the DNA molecule in a stable or transient manner.

“Vector” means a self-replicating nucleic acid molecule that transfers an inserted nucleic acid molecule into and/or between host cells. The term is intended to include vectors that function primarily for the replication of nucleic acid and expression vectors that function for transcription and/or translation of the DNA or RNA. Also intended are vectors that provide more than one of the above functions.

“Host cell” is intended to include any individual cell or cell culture that can be or have been recipients for vectors or the incorporation of exogenous nucleic acid molecules and/or proteins. It also is intended to include progeny of a single cell, and the progeny may not necessarily be completely identical (in morphology or in genomic or total DNA complement) to the original parent cell due to natural, accidental, or deliberate mutation.

An “antibody” is an immunoglobulin molecule capable of binding an antigen. As used herein, the term encompasses not only intact immunoglobulin molecules, but also anti-idiotypic antibodies, mutants, fragments, fusion proteins, humanized proteins and modifications of the immunoglobulin molecule that comprise an antigen recognition site of the required specificity.

An “antibody complex” is the combination of antibody (as defined above) and its binding partner or ligand.

A “suitable cell” for the purposes of this invention is one that includes, but is not limited to, a cell expressing Wnt or SFRP, e.g., a bone marrow cell, preferentially an hOB cell.

A “biological equivalent” of a nucleic acid molecule is defined herein as one possessing essential identity with the reference nucleic acid molecule. A fragment of the reference nucleic acid molecule is one example of a biological equivalent.

A “biological equivalent of a polypeptide or protein” is one that retains the same characteristic as the reference protein or polypeptide. This definition includes fragments of the reference protein or polypeptide that retain the same characteristic as the reference protein or polypeptide.

Proteins and polypeptides also can be obtained by chemical synthesis using a commercially available automated peptide synthesizer such as those manufactured by Applied Biosystems, Inc., (Foster City, Calif.) Model 430A or 431A. The synthesized protein or polypeptide can be precipitated and further purified, for example by high performance liquid chromatography (HPLC). Accordingly, this invention also provides a process for chemically synthesizing the proteins of this invention by providing the sequence of the protein and reagents, such as amino acids and enzymes and linking together the amino acids in the proper orientation and linear sequence.

Alternatively, the proteins and polypeptides can be obtained by well-known recombinant methods as described, for example, in Sambrook et al. ((1989) Molecular Cloning: A Laboratory Manual. 2d ed. Cold Spring Harbor Laboratory) using, for example, the host cell and vector systems described and exemplified in U.S. Patent Application, Pub. No. 2004/0115195 (U.S. Ser. No. 10/666,851). An SFRP, analog, mutein or fragment thereof, may be produced by growing a host cell containing a nucleic acid molecule encoding the desired protein, the nucleic acid being operatively linked to a promoter of RNA transcription. The desired protein may be introduced into the host cell by use of a gene construct which contains a promoter and termination sequence for the nucleic acid sequence of the desired protein. The host cell is grown under suitable conditions such that the nucleic acid is transcribed and translated into protein. In a separate embodiment, the protein is further purified.

In accordance with the present invention, there may be employed conventional molecular biology, microbiology, recombinant DNA, immunology, cell biology and other related techniques within the skill of the art. See, e.g., Sambrook et al. (2001) Molecular Cloning: A Laboratory Manual. 3^{rd} ed. Cold Spring Harbor Laboratory Press: Cold Spring Harbor, N.Y.; Sambrook et al. (1989) Molecular Cloning: A Laboratory Manual. 2^{nd} ed. Cold Spring Harbor Laboratory Press: Cold Spring Harbor, N.Y.; Ausubel et al. eds. (2006) Current Protocols in Molecular Biology. John Wiley and Sons, Inc.: Hoboken, N.J.; Bonifacino et al. eds. (2006) Current Protocols in Cell Biology. John Wiley and Sons, Inc.: Hoboken, N.J.; Coligan et al. eds. (2006) Current Protocols in Immunology, John Wiley and Sons, Inc. : Hoboken, N.J.; Coico et al. eds. (2006) Current Protocols in Microbiology, John Wiley and Sons, Inc. : Hoboken, N.J.; Coligan et al. eds. (2006) Current Protocols in Protein Science, John Wiley and Sons, Inc. : Hoboken, N.J.; Enna et al. eds. (2006) Current Protocols in Pharmacology John Wiley and Sons, Inc.: Hoboken, N.J.; Hames et al. eds. (1999) Protein Expression: A Practical Approach. Oxford University Press: Oxford; Freshney (2000) Culture of Animal Cells: A Manual of Basic Technique. 4^{th} ed. Wiley-Liss; among others. The Current Protocols listed above are updated several times every year.

The proteins of this invention also can be combined with various liquid phase carriers, such as sterile or aqueous solutions, pharmaceutically acceptable carriers, suspensions and emulsions. Examples of non-aqueous solvents include propyl ethylene glycol, polyethylene glycol and vegetable oils. When used to prepare antibodies, the carriers also can include an adjuvant that is useful to nonspecifically augment a specific immune response. A skilled artisan can easily determine whether an adjuvant is required and select one. However, for the purpose of illustration only, suitable adjuvants include, but are not limited to, Freund's Complete and Incomplete, mineral salts and polynucleotides.

### Therapeutic Applications

The methods of the present invention allow for identification of compounds that target Wnt signaling.

Since Wnts have been implicated as proto-oncogenes, SFRPs/SARPs may serve as tumor suppressors due to their ability to antagonize Wnt activity. As stated above, constitutively active Wnt signaling contributes to cancer. These proteins may also be utilized in tissue regeneration. For example, since FrzB-1 stimulated ectopic chondrogenic activity in vivo, it could be used to accelerate fracture repair or the healing of joints after hip and knee replacement (see, International Patent Publication No. WO 98/16641, incorporated herein by reference in its entirety). Finally, because SFRPs/SARPs appear to control apoptosis, these proteins could also be utilized to treat a variety of degenerative diseases including neurodegeneration, myodegeneration and osteodegeneration disorders.

Wnts may also be utilized in tissue regeneration. For example, since FrzB-1 stimulated ectopic chondrogenic activity in vivo, it could be used to accelerate fracture repair or the healing of joints after hip and knee replacement (see, International Patent Publication No. WO 98/16641, incorporated herein by reference in its entirety). Finally, because SFRPs/SARPs appear to control apoptosis, these proteins could also be utilized to treat a variety of degenerative diseases including neurodegeneration, myodegeneration and osteodegeneration disorders.

Pharmaceutical compositions of the test compounds discovered using the present invention are useful for treating or preventing osteodegeneration disorders such as osteoporosis and the bone resorptive disease, Paget's disease. For instance, when SFRP-1 expression and/or activity are abolished in vivo (e.g., in transgenic mice) bone density is increased, resulting in a delay of age-dependent bone loss (see WO 01/19855, incorporated herein by reference in its entirety). These effects correlate generally with an increased rate of bone formation, a decrease in osteoblast and osteoclast apoptosis, and an increase in osteoblast differentiation. Disruption of the fine balance between the differentiation of new osteoclast and osteoblast cells and the timing of cell death by apoptosis are thought to be important mechanisms behind bone loss disorders. Thus, therapeutic agents that alter the prevalence of apoptosis in osteoblasts and/or osteoclasts are useful and desirable to correct the imbalance in cell numbers that is the basis of diminished bone mass and increased risk of fractures in osteoporosis. For review, see, Manolagas (2000) Endocr. Rev. 21:115-137; and Weinstein and Manolagas (2000) Am. J. Med. 108:153-164.

For example, in one embodiment, test compounds discovered using the present invention may be used to prevent an osteodegenerative disorder, e.g., in an individual who may not have a bone degeneration disorder but who has or is suspecting of being susceptible to such a disorder. In another embodiment, the test compounds discovered using the present invention can be used to prevent Type II or “senile” osteoporosis. As a particular example, and not by way of limitation, test compounds discovered using the present invention may be administered to a juvenile, adolescent or young adult.

Altering the activity of SFRP-1 does not produce any significant side effects, e.g., on cortical bone and non-skeletal tissues, in body or organ weight; serum calcium, phosphorus, bone-alkaline phosphatase or osteocalcin levels; urinary deoxy-pyridinoline cross-link levels; total body bone mineral density (BMD), bone mineral content and percentage body fat; or cortical BMD (see U.S. Patent Application Pub. No. 2004/0115195, U.S. Ser. No. 10/666,851). Even though the inhibition of SFRP-1 may increase bone density, it does not alter skeletal development. Consequently, therapeutic methods and compositions that specifically inhibit SFRP-1 activity and/or expression are expected to have very few or even no detrimental side effects.

Alternatively the test compounds discovered through the use of the methods of the present invention can be used to treat diseases, such as osteopetrosis and osteosclerosis that are the result of aberrant bone formation or abnormal increases in bone formation. Such diseases may be treated by disrupting or decreasing Wnt activity by means of, for example, antibodies, antisense nucleotides, siRNAs or shRNAs that inhibit expression, or small molecule inhibitors that disrupt or decrease activity and/or expression.

### Pharmaceutical Compositions

A “pharmaceutical composition” is intended to include antibodies, small molecules, or test compounds that are targeted to particular amino acids for decreasing or blocking activity as the active agent with a carrier, inert or active, making the composition suitable for diagnostic or therapeutic use in vitro, in vivo, or ex vivo.

This invention also provides compositions containing a test compound discovered using the present invention and an acceptable solid or liquid, carrier buffer, or diluent. An effective amount of one or more active ingredient is used which is sufficient to accomplish the desired regulatory effect on a bone-forming activity or apoptosis activity. An effective amount can be determined by conventional dose-response curves for the desired activity. When the compositions are used pharmaceutically, they are combined with a “pharmaceutically acceptable carrier” for diagnostic and therapeutic use. The formulation of such compositions is well known to persons skilled in this field. Pharmaceutical compositions of the invention may comprise one or more additional active components and include a pharmaceutically acceptable carrier. The additional active component may be provided to work in combination with an active component. In alternative embodiments, the additional active component is added because it works on the same disease or disorder, or the additional active may work on other diseases or disorders present in a human or animal.

Suitable pharmaceutically acceptable carriers and/or diluents include any and all conventional solvents, dispersion media, fillers, solid carriers, aqueous solutions, coatings, antibacterial and antifungal agents, isotonic and absorption delaying agents, and the like with which the compound is administered. The term “pharmaceutically acceptable carrier” refers to a carrier that does not cause an allergic reaction or other untoward effect in patients to whom it is administered. As used herein, the term “pharmaceutically acceptable” means approved by a regulatory agency of the Federal or state government or listed in the U.S. Pharmacopeia or other generally recognized pharmacopeia for use in animals, and more particularly, in humans. Specific, suitable pharmaceutically acceptable carriers include, but are not limited to, for example, one or more of water, saline, phosphate buffered saline, dextrose, glycerol, ethanol and the like, as well as combinations thereof. Water or aqueous solution saline solutions and aqueous dextrose and glycerol solutions may be employed as carriers, particularly for injectable solutions. Pharmaceutically acceptable carriers may further comprise minor amounts of auxiliary substances such as wetting or emulsifying agents, preservatives or buffers, which enhance the shelf life or effectiveness of one or more of the active components of the composition. The use of such media and agents for pharmaceutically active substances is well known in the art and suitable pharmaceutical carriers are described in “Remington's Pharmaceutical Sciences” by E. W. Martin. Except insofar as any conventional media or agent is incompatible with the active ingredient, use thereof in immunogenic compositions of the present invention is contemplated.

These pharmaceutical compositions also can be used for the preparation of medicaments for the diagnosis and treatment of pathologies associated with neurodegenerative (i.e., Huntington's disease, Alzheimer's disease, and spinal cord injuries), myodegenerative (i.e., muscular dystrophy, myasthenia gravis, myotonic myopathies) and osteodegenerative disorders (i.e., osteoporosis). These compositions can also be used for the preparation for medicaments for the diagnosis and treatment of diseases such as Paget's disease, osteosclerosis, osteogenesis imperfecta, fibrous dysplasia, hypophosphatasia and osteopetrosis.

Antibodies for in vivo use may recognize a topological or conformational epitope present on the Wnt-Fzd molecule. The antibodies contemplated by the present invention may or may not recognize denatured Wnt-Fzd or Wnt-Fzd fragments. Polyclonal and monoclonal antibodies can be prepared by conventional methods, e.g., by immunization with Wnt-Fzd or protein or a mutant thereof. Alternatively, an antibody is raised against an amino acid sequence (a) that is specific to a polypeptide/protein (or proteins) and (b) that is also more likely to be antigenic. One can select a sequence specific for a protein by performing sequence analysis and using any conventional programs for sequence alignment and sequence comparisons. An amino acid sequence that is hydrophilic at one or more ends, or at both ends, is generally favored for raising antibodies. In addition to employing amino acids that are hydrophilic, in some embodiments the hydrophilic amino acids are also basic (non-acidic). One can also employ any amino acid that increases antigenicity. For example, often prolines are employed in the center portion of the sequence. Antigenicity can be measured by an increase in the decrease in the amount of antibody that is produced when generating antibodies against an initial test sequence, which is specific to particular protein(s).

In certain embodiments of the present invention, the antibody is raised against a sequence comprising at least 8 consecutive amino acids of a Wnt-Fzd protein(s), or a sequence comprising at least 10 consecutive amino acids of a Wnt-Fzd protein(s). In other embodiments, the antibody is raised against amino acid sequence comprising about 15 to about 30 amino acids.

The compositions of the test compounds discovered using the methods of the present invention can be administered to an individual in need of facilitated neural, muscle, cartilage, and bone growth by numerous routes, including, but not limited to, intravenous, subcutaneous, intramuscular, intrathecal, intracranial and topical. The composition may be administered directly to an organ or to organ cells by in vivo or ex vivo methods.

These compositions may be in soluble or microparticular form or may be incorporated into microspheres or microvesicles, including micelles and liposomes.

### Screening Methods

Screening methods may be devised wherein the amount of a test compound bound to a protein may be determined. In such a screening method, there may be two samples. In one, a molecular species, a “test compound,” and wild-type Wnt/Fzd chimeric protein may be incubated together, and, separately in another, the test compound and a Wnt-Fzd mutant may be incubated together. The test compound may be, but is not limited to, a small molecule, polypeptide, or nucleic acid or may be a gene that is knocked-out or knocked-down in a cell. The amount of compound bound to each may then be determined using any of various methods known to those of ordinary skill in the art. These could include, but are not limited to, using fluorescence (such as in a FRET-based assay), nuclear magnetic resonance, and chromatography.

Screening methods may be devised based on the specific domains or amino acids found to be important in inhibition of the Wnt signaling pathway. As non-limiting examples, the described assay methods may be used to determine a change in Wnt signaling in the presence or absence of a test compound. Any other assay method may be used provided that it shows a change in Wnt signaling due to the presence or absence of a test compound. Such assays may be, but are not limited to, activity or binding assays.

Suitable cells can be used for preparing diagnostic assays, for expression or for preparing nucleotide-based diagnostic kits. The cells may be made or derived from yeast, bacteria, fungi, or viruses. In certain embodiments, the cells are hOB cells, in particular a novel immortalized pre-osteocytic cell line referred to as hOB-01-C1-PS-09 cells (which are deposited with American Type Culture Collection in Manassas, Va. with the designation PTA-785), and osteoblast cells having the identifying characteristics of hOB-01-C1-PS-09 cells as well as osteoblast cells made therefrom, e.g. progeny.

Agents according to the present invention may be identified by screening in high-throughput assays, including, without limitation, cell-based or cell-free assays. It will be appreciated by those skilled in the art that different types of assays can be used to detect different types of agents. Several methods of automated assays have been developed in recent years so as to permit screening of tens of thousands of compounds in a short period of time (see, e.g., U.S. Pat. Nos. 5,585,277; 5,679,582; and 6,020,141).

### Transgenic Animals Demonstrate Relationship between SFRP and Bone Formation

A compound that inhibits osteoblast/osteocyte apoptosis would conceivably be an anabolic bone agent by prolonging the lives of these cells and thereby either increasing the amount of bone matrix that is synthesized and mineralized and/or maintaining the integrity of the bone. In order to test this hypothesis and determine if SFRP-1/FRP-1/SARP-2 affects the skeleton, SFRP-1 −/− mice were prepared in WO 01/19855 (see also Wattler et al. (1999) BioTechniques 26:1150-1160). Deleting the SFRP-1/FRP-1/SARP-2 gene from mice would be akin to inhibiting its function with a compound, and this process allows validation of this gene/protein as a potential pharmaceutical target for osteoporosis.

The SFRP-1 knock-out mice were generated in WO 01/19855 by substituting exon 1 of the mouse SFRP-1 gene with β-galactosidase reporter gene/neomycin resistance gene expression cassette. Northern blot analysis of poly A+RNA isolated from either female of male kidneys (age 16-18 weeks) demonstrated high levels of SFRP-1 mRNA expression (4.4 kb) in the wild-type (WT) control mice, but a complete absence of gene expression in the knock-out (KO) mice.

Micro computerized tomography (micro-CT) was used in WO 01/19855 to characterize the trabecular bone architecture of the distal femurs from male and female wild-type control (+/+) and knock-out (−/−) mice (for a review of this technique, see Genant et al. (1999) Bone 25: 149-152 and Odgaard (1997) Bone 20:315-328). In the 20 week old males, the −/− mice had 31% more trabecular bone volume (BV/TV) and an 8% increase in trabecular thickness (Tb. Th.) when compared to the +/+control mice. In the 26-27 week old females, the −/− mice had a 91% increase in trabecular connectivity density (Conn. Den.), a 16% increase in trabecular number (Tb. N.) and a 16% decrease in trabecular spacing (Tb. Sp.) when compared to the +/+ control mice.

Introduction of the constitutively active Wnt-Fzd chimera, especially the Wnt3-Fzd1 chimera, gene in mice is expected to lend to increased parameters of trabecular bone formation (P. J. Meunier (1995) Bone Histomorphometry, in Osteoporosis: Etiology, Diagnosis, and Management. 2 ed. B. L. Riggs and L. J. Meltonlil, eds. Lippincott-Raven: Philadelphia, pages 299-318).

Transgenic and/or non-genetically modified animals may be used for methods of screening compounds which may be of pharmaceutical interest. Non-limiting examples of animals would be a transgenic animal genetically modified to express a constitutively active or inactive Wnt-Fzd chimera, especially a Wnt3-Fzd1 chimera. A test compound may be administered to one or more of these types of animals and the resulting phenotype compared to that of an identical animal to which was administered a placebo. If there is a change in a bone formation parameter (as described above) of the animal administered the test compound as compared to either an identical animal administered a placebo, then the test compound modulates the Wnt pathway by way of the SFRP protein molecule through the amino acid(s) which was/were mutated.

### EXAMPLE

The present invention is next described by means of the following example. However, the use of this and other examples anywhere in the specification is illustrative only and in no way limits the scope and meaning of the invention or of any exemplified form.

### Materials and Methods

Isolation of Wnt3-FZ1 chimera plasmid: The Wnt3-Fz1 chimera contains N-terminal Wnt3 with a HA tag, a 32 amino acid glycine spacer and a mature Fz1 receptor sequence. The plasmid was generated in steps and is as follows: The glycine spacer was first synthesized using PCR amplification of a purified oligo with the sequence ACCGGTACCGGGCCCGGAGGCGGGGGCGGAGGGGGCGGCGGGGGCGGAGGGGGCT CCACCGGTCCCGAATTCAAA (SEQ ID NO: 32) with the following primer pairs 5′ ACCGGTACCGGGCCCGGA (SEQ ID NO: 33) and 3′ TTTGAATTCGGGACCGGTGGATCCCCCT (SEQ ID NO: 34) and 5′ ACCAGATCTGGGCCCGGAGGC (SEQ ID NO: 35) and 3′ primer same as above. The purified PCR products were digested with ASP718 and BamHI; BglII and EcoRI, respectively, and the gel-purified fragments were ligated into Asp718 and EcoRI site of pcDNA3.1(+) vector. The Wnt3 ORF was PCR amplified using 5′ primer ATAGCTAGCCCACCATGGAGCCCCACCTGCTCGGGCTG (SEQ ID NO: 36) and 3′ primer TTTCTAGAGTTAAAGCTTAGGCCCTGGACCCAAAGAAG (SEQ ID NO: 37) and using WN3-HA tagged plasmid as the template (Upstate Biotechnology, Lake Placid, N.Y.). The purified PCR product was digested with NheI and HindIII and cloned into corresponding sites in Glycine spacer plasmid. The FZI ORF with out the signal sequence was amplified from FZ1 plasmid using the following primers: 5′ TAAACCGGTGGGCCAGGCCAGGGGC (SEQ ID NO: 38) and 3′ GGGCCCTCTAGACTCGAGTCAGAC (SEQ ID NO: 39). The purified PCR amplified product was digested with AgeI and XbaI and was cloned into the corresponding sites in Wnt3-Glycine spacer plasmid to generate Wnt3-FZ1 chimera plasmid.

The Wnt3-FZ1 chimera with deletion of cytoplasmic region was generated by PCR amplifying the carboxyl region of FZ1 with the following primers 5′ GATGGATCCAAGACCGAGAAGCTGGA (SEQ ID NO: 40) and 3′ primer ATTTCTAGAATTAGGGTGACCAGATCCCAGAAGCCCGAC (SEQ ID NO: 41) and cloning the FspI and XbaI fragment into the corresponding site of Wnt3-FZ1 chimera plasmid. The deletion plasmid has a proline residue in place of glycine and has a unique Bst E II restriction site, which was subsequently used in the isolation of cytoplasmic mutants of Wnt3-FZ1 chimera. The mutations with in the cytoplasmic portion of FZ1 was generating by PCR amplifying the cytoplasmic region with 5′ PCR primers with BstEII restriction site and desired changes in the codon sequence for amino acid changes and the 3′ primer with the XbaI site and cloning the PCR amplified product into BstEII and Xba I site of Wnt3-FZ1 del cytoplasmic plasmid.

Mutations in the CRD domain of Wnt3-FZ1 chimera: A Unique Eco RV site in the 2^{nd} cysteine loop of CRD domain was generated by PCR amplification of the CRD domain using the following primers: 5′ primer GGAGGGGGATCCACCGGTGGGCCA (SEQ ID NO: 42) and 3′ primer CGCGATATCCGTGCACAGCGGGAT (SEQ ID NO: 43) and 5′ CTGTGCACGGATATCGCGTAC (SEQ ID NO: 44) and 3′ CCGGGTAGCTGAAGCGCCGCATG (SEQ ID NO: 45) primers. The PCR products were digested with BamHI and EcoRV; EcoRV and DraIII, respectively, and the two PCR fragments were ligated to Bam HI and Dra III fragment of Wnt3-Fz1 chimera plasmid. The 29 amino acid deletion mutant was generated by PCR amplifying the FZ CRD domain using the following primers 5′ ACGGATATCGTGAAAGTGCAGTGTTCCGCTG (SEQ ID NO: 46) and 3′ primer CCGGGTAGCTGAAGCGCCGCATG (SEQ ID NO: 45) and the EcoRV and DraIII fragment was cloned into the EcoRV-DraIII fragment of modified Wnt3-FZ1 chimera. The mutation of tyrosine and asparagine to alanine were generated by PCR amplifying the FZ 1 CRD portion using 5′ primers with EcoRV site and desired change in the codon sequence and 3′ primer with the DraIII site and cloning the amplified fragment to EcoRV and DraIII sites of modified Wnt3-FZ1 chimera plasmid.

The nucleotide sequences of all the plasmids were verified by sequencing.

Ad5 recombinant virus expressing Wnt3-Fz1 chimera: The transcription unit of the chimera protein containing the CMV promoter and Wnt3-Fz1 fusion protein reading frame and its mutants as a MluI-XbaI fragment were cloned into the corresponding sites of modified pENTR 1A vector (Invitrogen™) containing SV40 PolyA signal sequence. In vitro recombination of the above plasmids with the pAD PL/Dest vector (Invitrogen™) resulted in plasmids suitable for generation of recombinant adenovirus. The plasmids were digested with the PacI enzyme to release the vector part and the purified DNAs were transfected into 293 A cells using Lipofectamine™ (Invitrogen™). The cells were overlaid with agarose and the plaques were isolated and amplified. The viruses were plaque purified and virus stocks were prepared and titered in 293 A cells.

Cell line and Transfection: The osteosarcoma cell line U2OS (ATCC) was maintained in growth media consisting of McCoy's 5A medium (Invitrogen™) containing 10% fetal calf serum (Hyclone, Logan Utah.), 2 mM Glutamax-1™ (Invitrogen™) and 1× penicillin and streptomycin (Invitrogen™) and incubated at 37° C. with 5% CO₂/95% humidified air. For transfection studies, the cells were plated in a 96-well tissue culture plate in growth media without antibiotics and incubated overnight in the incubator. The growth medium was removed, and the cells were washed once with OPTI-MEM® I medium (Invitrogen™) and were then fed with 100 ul of OPTI-MEM® I. The cells were transfected with the following DNA's using Lipofectamine™ as recommended by the manufacturer (Invitrogen™). For each transfection, the following DNA's were diluted together in OPTI-MEM® I medium: (100 ng of 16×TCF-Luciferase, 20 ng of Wnt 3 (Upstate Biotechnology, Lake Placid, N.Y.), 1-5 ng of Wnt3-FZ1 chimera or its mutants and 25 ng of β-Galactosidase (Clontech, Palo Alto, Calif.) and 0.4 ul of Lipofectamine™ 2000 (Invitrogen™, Carlsbad, Calif.) in a total volume of 50 ul. The DNA-Lipofectamine™ mixture was then added to each well and the plates were incubated in a 37° C. incubator for 4 hours. The medium was then removed and the cells were washed with 150 ul of phenol red free RPMI 1640 medium (Invitrogen™), re-fed with 100 ul of RPMI medium supplemented with 2% fetal calf serum, 2 mM Glutamax-1™ and 1% penicillin-streptomycin, and incubated in a 37° C. incubator overnight. The next day, the cells were washed twice with 150 ul/well of PBS without Ca++ and Mg++ (Invitrogen™) and then lysed with 50 ul/well of cell culture lysis reagent (Promega, Madison, Wis.). The cell lysates were assayed for Luciferase (Promega) and β-galactosidase (Tropixo, Bedford, Mass.) activity using a microlumatPLUS luminometer (EG&G Berth hold). The luciferase activity was normalized with β-gal to offset the transfection efficiency and the data was analyzed using the JMP® program (SAS Institute). Activation of Wnt signaling is presented as fold activation over the control containing TCF-Luciferase reporter.

Wnt-Fz chimera virus upregulates Wnt signaling: For infection studies, the cells were plated in a 96-well tissue culture plate in growth media and incubated overnight in the incubator. The growth medium was removed, and the cells were infected with 10 PFU of Ad5 Wnt3 and 50 PFU of 16×TCF-Luciferase in 50 ul of media containing 2% serum. After 1 hr incubation, the virus inoculum was removed and the cells were fed with 100 ul of the growth media and incubated in a 37° C. incubator overnight. The next day, the cells were washed twice with 150 ul/well of PBS without Ca++ and Mg++ (Invitrogen™) and then lysed with 50 ul/well of cell culture lysis reagent (Promega) and assayed for luciferase activity as described above.

Differentiation of C3H10T1/2 cells into osteoblasts and adipocytes: C3H10T1/2 mesenchymal stem cells were plated at 3.16E+4 cells/cm² in T225 flasks in Dulbecco's Modified Eagle Medium (DMEM; Invitrogen™, Grand Island, N.Y.) containing 10% heat inactivated fetal bovine serum (Invitrogen™), 4500 mg/liter glucose, 1× Glutamax-1™ Supplement (2 mM L-Alanyl-L-Glutamine; Invitrogen™), 1 mM Sodium Pyruvate (Invitrogen™), and 1× Penicillin/Streptomycin solution (Invitrogen™) (Growth Medium). The cells were incubated overnight at 37° C. inside a 5% CO₂/95% humidified air incubator. After 24 hours in culture, the medium was removed, and the cells were infected with Ad5 WNT3/FZ1, Ad5 WNT3/FZ1 cytoplasmic tail deletion, or Ad5 CMV-Bgal (infection control) at 400 MOI in growth medium for 1 hour at 37° C. A mock infection was also performed using virus-free growth medium. After one hour of infection, the virus inoculum was removed, and the cells were washed once with serum-free DMEM, re-fed with fresh growth medium, and incubated at 37° C. for 4 hours. After the four hour recovery period, the infected and mock-infected cells were trypsinized, counted, and plated in 24-well tissue culture plates at 4E+4 cells/cm² in growth medium containing 50 ug/ml L-Ascorbic Acid Phosphate (Wako, Richmond, Va.), 10 mM B-glycerol phosphate (Sigma, St. Louis, Mo.) and 100 nM Menadione sodium bisulfite (Vitamin K3; Sigma) (Differentiation Medium). The cells were incubated at 37° C. inside a 5% CO₂/95% humidified air incubator. The differentiation medium was replaced every three to four days. For alkaline phosphatase assay, the cells were washed with PBS and lysed and the cell lysates were assayed for total protein and alkaline phosphatase activity. For adipogenic differentiation, the cells were infected and plated as above and the cells were treated with adipogenic media containing insulin (10 ug/ml,) dexamethasone (1 uM) and IBMX (0.5 mM). After 3 days, the media was replaced with adipogenic progression medium (growth media with 10 ug/ml insulin), with subsequent progression media replacement every third day. 15 days post-infection, the cells were stained with Oil-Red-O stain.

### Results

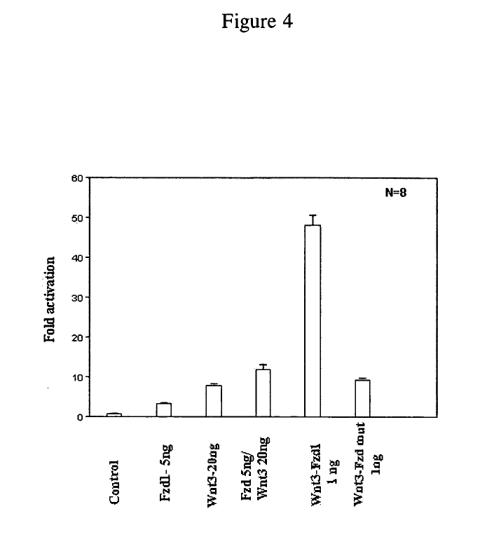

The Wnt3-FZ1 chimera is more efficient than Wnt3 in activating canonical Wnt signaling (FIG. 4): The U2OS cells contain all the components necessary for the canonical Wnt signaling and express both frizzled receptors and Wnt. The optimized TCF-Luciferase reporter containing 16 copies of TCF element upstream of minimal tk promoter is used as a measure of Wnt signaling. Transfection of U2OS cells with Wnt cDNA results in about 8-10 fold increase in luciferase activity compared to control. Transfection of U2OS cells with the frizzled 1 expression plasmid resulted in about 3 fold increase in activity suggesting the presence of endogenous Wnt. Co-transfection of Wnt and Frizzled resulted in synergistic activation, suggesting that both the endogenous and expressed Fz1 receptor is being utilized in the activation of TCF-luciferase reporter. Transfection of Wnt3-FZ1 chimera resulted in about 50-60 fold activation of TCF-Luciferase reporter and the response obtained with transfection of 1 ng of Wnt3-FZ1 chimera plasmid is nearly 4-5 times better than the response obtained with 20 ng of Wnt3 expression plasmid. The results clearly demonstrate that the Wnt3-FZ1 chimera is much more efficient than Wnt expression alone in activating the canonical Wnt signaling.

A dose response study with Wnt3-FZ1 chimera has shown optimal signaling at 1 ng/well and further increase in the amount of plasmid DNA did not increase the response further, rather it decreased the response at 10-20 ng of transfected DNA. For rest of the experiment 1 ng/well of the Wnt-Fz1 chimera was used.

The cytoplasmic domain is critical for Wnt signaling (FIG. 5): In order to determine the role of cytoplasmic tail of Fz1 in the Wnt signaling, a mutant of Wnt3-Fz1 with the deletion of cytoplasmic tail was generated and tested for its ability to active the TCF-Luciferase reporter. The cytoplasmic deletion mutant showed 8 fold less activity compared to Wnt3-Fz1 chimera clearly indicating that the cytoplasmic tail plays a critical role in the FZ1 mediated Wnt signaling. A single amino acid change from glycine to proline in the amino-terminal portion of the cytoplamic tail that generates the unique BstE II restriction site that was used to generate mutants of Wnt3-FZ1 cytoplasmic mutants and resulted in a slight increase in the activity. It has been shown that FZ1 cytoplasmic tail contains two PDZ binding domains, the N-terminal KTXXXW (SEQ ID NO: 47) is conserved in all Frizzled receptors and a terminal ETTV (SEQ ID NO: 48) binding domain. The Wnt3-Fz1 chimeras with the mutation of two PDZ binding domains were assayed for their ability to activate canonical Wnt signaling. Mutation of ETTV (SEQ ID NO: 48) to GAAA (SEQ ID NO: 49) in Wnt3-FZ1 chimera did not affect its activity; where as mutation of KTXXXW (SEQ ID NO: 47) resulted in substantial loss of activity. Individual mutation of either KT to AA or combined mutation into AAXXXA (SEQ ID NO: 50) resulted in 50% loss of activity indicating that KTXXXW (SEQ ID NO: 47) in Fz1 cytoplasmic domain is critical in the activation of Wnt signaling. A mutation of both the PDZ binding domain resulted in 80% activity suggesting that signaling involving both the PDZ binding domain has a synergistic effect.

The FZ1 CRD domain is necessary and the tyrosine and asparagines residues in the CRD is critical for Wnt signaling (FIGS. 3 and 7): The interaction of Wnt with the CRD domains of FZ has been well documented and is shown to be critical for Wnt signaling. Mutation studies with the SFRP-1 CRD domain have shown that 2^{nd} loop of the CRD is critical and particularly the tyrosine residue is necessary for the Wnt antagonist function of SFRPs. A tyrosine and asparagine residue is conserved in all the FZ CRD and a tyrosine is conserved in SFRP-1, 2, 5 and is replaced by tryptophan in SFRP-3 and 4. A mutation study with SFRP-1 has shown that tyrosine residue is critical for its Wnt antagonist function, and its mutation to alanine, aspartate of asparagines results in substantial loss of Wnt antagonist function. In order to address the role of FZ1 CRD domain, a deletion of 29 amino acid with in the 2^{nd} cysteine loop is generated and tested for its ability to activate TCF-luciferase reporter. Wnt3-FZ1 chimera with the deletion failed to activate the TCF-Luciferase reporter suggesting that the 2^{nd} cysteine loop is critical for Wnt signaling. In order to determine the role of conserved tyrosine and asparagine residues, they were mutated either individually or together into alanine residues. The tyrosine to alanine mutation in Wnt-Fz1 chimera CRD resulted in almost 95% loss of canonical Wnt signaling and mutation of asparagines into alanine resulted in 80% loss of activity. The Wnt3-Fz1 chimera with tyrosine and asparagines mutation into alanine residues is totally inactive in up regulating the TCF-Luciferase reporter. These results confirm that the highly conserved amino cid tyrosine and asparagines with in the 2^{nd} loop of FZ CRD is critical for Wnt mediated activation of canonical signaling.

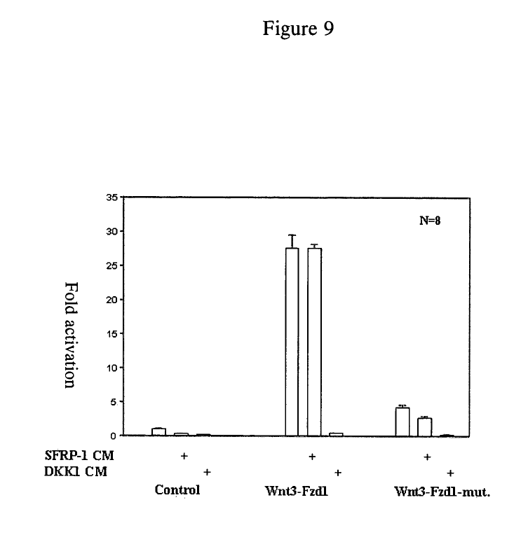

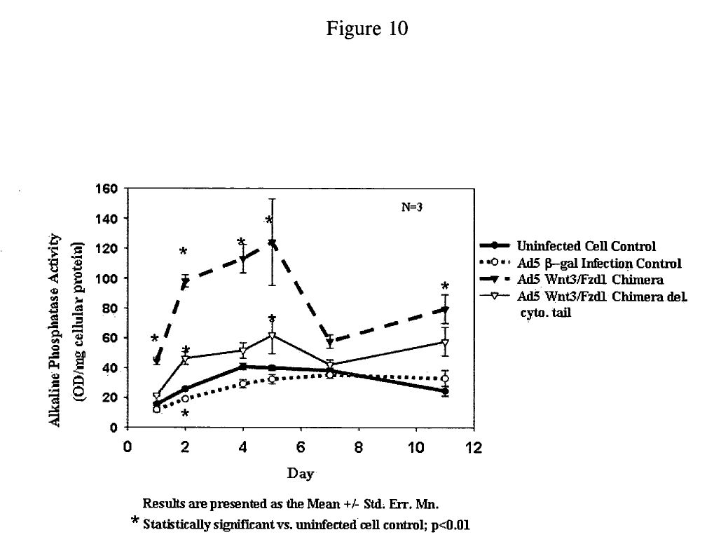

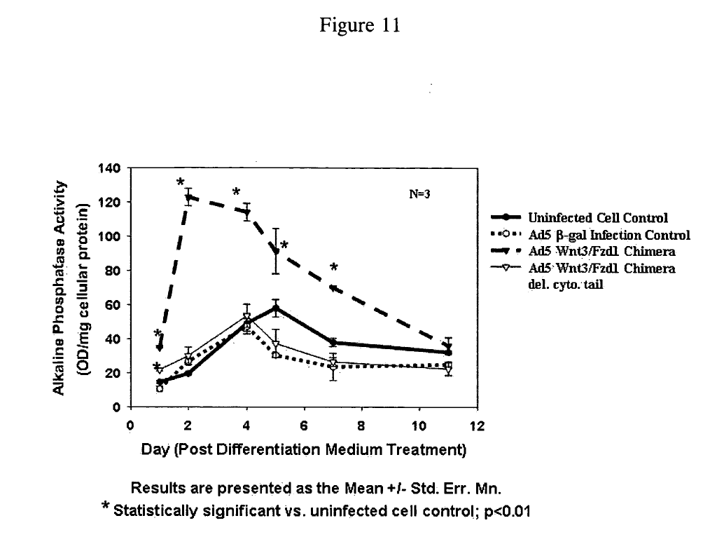

DKK-1 totally abolishes the activity of Wnt3-Fz1 chimera (FIGS. 5 and 9): The Ad5 recombinant expressing the Wnt3-Fz1 chimera activates Wnt signaling as measured by a 25-30-fold increase in the TCF-Luciferase activity. Dkk-1 and SFRPs antagonize the wnt signaling by interfering with the interaction of Wnt with LRP and Wnt with the Fz1 receptor respectively. Addition of the DKK-1 rich conditioned media totally abolishes the Wnt signaling. The data clearly suggest the Wnt3-Fz1 chimera interacts with the LRP to mediate the Wnt signaling. However SFRP-1 protein fails to inactivate the Wnt-Fz1 chimera action, possibly due to the close proximity of the Wnt3 and FZ in the chimera. Co-culture assay using the cells infected with TCF-reporter and Wnt3-Fz1 virus fails to activate the Wnt signaling where as the cells infected with both the viruses' upregulate the luciferase activity. The above results indicates that Wnt3 portion of the in the chimera acts in cis and fails to activate the endogenous Fz1 receptor. Wnt3-Fz1 chimera is a potent activator of differentiation of C3H10T1/2 cells into osteoblasts: C3H10T1/2 cell is a murine embryonic mesenchymal cell, which retains the potential of differentiating into osteoblast. Expression of Wnt3-Fz1 chimera results in increased alkaline phosphatase activity as early as day 1 and peaks on day 2 with ˜12 fold increase compared to the uninfected or Ad5 β-gal infected control cells (see FIGS. 10 and 11). Contrary, expression of the Wnt3-Fz1 chimera results in the inhibition of differentiation of cells into adipocytes. These results suggest that activation of Wnt signaling by expressing that wnt3-Fz1 chimera results in the differentiation of the C3H10T1/2 cells into osteoblasts.

### Discussion

The Wnt-Fz fusion protein has been shown to activate canonical wnt signaling and it has been proposed that the main function of Wnt is to nucleate the formation of physical complex between LRP and a frizzled molecule. Previous studies have generally used xenopus Wnt and various Fz receptors. In the present work we have used human Wnt3 linked to Fz1 receptor and using an optimized TCF-luciferase reporter, we show a robust upregulation of wnt signaling with 30-50-fold increase in luciferase activity. The development replication defective adenovirus expressing Wnt-Fz1 chimera and the 16×TCF-Luciferase reporter has enabled us to study the wnt signaling in several cells line that are difficult to transfect. The DKK protein blocks the signaling of Wnt3-Fz chimera confirming that the interaction with LRP is essential for Wnt signaling.

All Fz receptors contain with in their extra cellular portion a region called Cysteine-rich domain, named for its invariant pattern of 10 cysteine residues. The CRD of frizzled has been crystallized and it binds to Wnt protein with nanomolar affinity. Several mutant in the Fz CRD has been engineered that effects Wnt binding suggesting that the interaction of Wnt with Fz CRD is critical for it s signaling. Contrary to this notion, Fz transgenes lacking the CRD were reported to respond normally to Wg and activate Arm signaling in vivo. However in the recent study, mutants of Fz CRD were not functional in cell culture or fully active in vivo. More over replacing the CRD with a structurally distinct wnt-binding domain reconstitute a Wg receptor. Based on the study, it has been proposed that the function of CRD is to bring the Wg in close proximity with the membrane portion of the receptor. In the present study, the 2^{nd} cysteine loop of the cytoplasmic portion of the CRD is critical for wnt signaling as a deletion of 29 amino acids with the loop totally abolishes the wnt signaling. More over change of two residues conserved in all frizzled receptor, tyrosine and asparagines to alanine results in total loss of Wnt signaling. The tyrosine residue is conserved in SFRP family members 1, 2 and 5 and is replaced by tryptophan in SFRP-2 and SFRP-3. The change of tyrosine tryptophan has no significant effect on SFRP-1 function, whereas, change of tyrosine to serine, alanine, aspartic resulted in about 40% loss of its wnt antagonist activity (unpublished results) clearly suggesting that the tyrosine residue with the 2^{nd} loop of CRD is critical for its wnt antagonist function. In the crystal structure of FZ CRD, tyrosine residue is buried with in the molecule and appears to keep the two helices apart. It is unlikely that tyrosine is a target for phosphorylation, as its change to tryptophan has no deleterious effect in SFRP-1 and change to phenylalanine results in slight loss of activity. We propose that the tyrosine is essential to keep the tertiary structure intact probably through its bulk side chain that keep the two helices separate and prevents from collapsing and stabilization of the structure possibly through hydrogen bonding.

Numerous studies suggest that activation of the Wnt/β-catenin pathway plays an important role in human tumorigenesis. Over expression of wnt has been observed in a variety of cancer cell lines including non small-cell lung cancer, mesothelioma, breast cancer and sarcomas. In animal models over expression of Wnt leads to tumors. The wnt-1 antisense RNA or monoclonal anti wnt-1 antibody is shown to reduce the Wnt signaling and reduce the tumor growth in vivo. Similarly, anti-wnt2 monoclonal antibody inhibited tumor growth in malignant melanomas. These findings hold promise that inhibition of wnt and Fz are targets for therapeutic intervention.

In the present study using an optimized TCF-Luciferase reporter system and expression of Wnt-Fz chimera through adenovirus, we demonstrate that Wnt-Fz chimera upregulates the canonical wnt signaling by about 30 folds. Such a cell-based assay can be used to identify inhibitors of wnt signaling that target the Wnt, frizzled and also downstream components of wnt signaling. Such an inhibitor has the therapeutic value to treat variety of cancers.

Several wnts including wnt5a, wnt 1 and wnt 7b has been shown to be present in fibroblast like synoviocytes and wnt signaling has been implicated in rheumatoid arthritis pathogenesis. Wnt and Fz receptor antagonists, or small molecule inhibitors of Wnt-Fz signaling may be useful for therapeutic intervention in refractory rheumatoid arthritis.

The wnt signaling plays an important role in the in the regulation of the bone mineral density. Several studies have demonstrated that wnts stimulate osteoblast precursor growth and their differentiation into osteoblasts. Wnt signaling is also shown to inhibit adipogenesis. In the present study, the adenoviral-mediated expression of wnt3-Fz chimera in C3H10T1/2 cells resulted in the inhibition of adipogenesis and activated the differentiation cells into osteoblast lineage. The Wnt-Fz chimera approach has the potential to be valuable tool to understand the mechanism and to identify the key players in the differentiation of stem cells.

Numerous references, including patents, patent applications and various publications, are cited and discussed in the description of this invention. The citation and/or discussion of such references is provided merely to clarify the description of this invention and is not an admission that any such reference is “prior art” to the invention described herein. All references cited and discussed in this specification are incorporated herein by reference in their entirety and to the same extent as if each reference was individually incorporated by reference.

What is claimed is:

1. A chimeric protein comprising a Wnt3 protein or portion thereof and a Fzd1 protein or portion thereof.

2. The chimeric protein of claim 1, wherein said Fzd1 protein or portion thereof comprises a modified cytoplasmic tail.

3. The chimeric protein of claim 1, wherein said Fzd1 protein or portion thereof does not comprise a cytoplasmic tail.

4. The chimeric protein of claim 1, wherein said Fzd1 protein or portion thereof comprises the amino acid sequence of SEQ ID NO: 11.

5. The chimeric protein of claim 1, wherein said Wnt3 protein or portion thereof comprises/consists of the amino acid sequence of SEQ ID NO: 1.

6. The chimeric protein of claim 1, wherein said Fzd1 protein or portion thereof comprises a modified cysteine-rich domain (CRD).

7. A nucleic acid comprising a sequence encoding any one of the chimeric proteins of claims 1-6.

8. An expression vector comprising the nucleic acid of claim 7.

9. A host cell transfected with the expression vector of claim 8.

10. A method assaying Wnt signaling, comprising:
(i) transfecting experimental cells with the expression vector of claim 8;
(ii) determining Wnt signaling of said experimental cells;
(iii) comparing the Wnt signaling of said experimental cells to the Wnt signaling of control cells,
thereby assaying Wnt signaling of said experimental cells.


SEQUENCE LISTING (50 sequences)

SEQ ID NO: 1 - protein, 373 aa, Homo sapiens
MEPHLLGLLLGLLLGGTRVLAGYPIWWSLALGQQYTSLGSQPLLCGSIPGLVPKQLRFCRNYIEIMPSVAEGVKLGIQECQHQFRGRRWNCTTIDDSLAIFGPVLDKATRESAFVHAIASAGVAFAVTRSCAEGTSTICGCDSHHKGPPGEGWKWGGCSEDADFGVLVSREFADARENRPDARSAMNKHNNEAGRTTILDHMHLKCKCHGLSGSCEVKTCWWAQPDFRAIGDFLKDKYDSASEMVVEKHRESRGWVETLRAKYALFKPPTERDLVYYENSPNFCEPNPETGSFGTRDRTCNVTSHGIDGCDLLCCGRGHNTRTEKRKEKCHCVFHWCCYVSCQECIRIYDVHTCKSMAYPYDVPDYASLGPGP

SEQ ID NO: 2 - protein, 388 aa, Homo sapiens
MGLWALLPSWVSTTLLLALTALPAALAANSSGRWWGIVNIASSTNLLTDSKSLQLVLEPSLQLLSRKQRRLIRQNPGILHSVSGGLQSAVRECKWQFRNRRWNCPTAPGPHLFGKIVNRGCRETAFIFAITSAGVTHSVARSCSEGSIESCTCDYRRRGPGGPDWHWGGCSDNIDFGRLFGREFVDSGEKGRDLRFLMNLHNNEAGRTTVFSEMRQECKCHGMSGSCTVRTCWMRLPTLRAVGDVLRDRFDGASRVLYGNRGSNRASRAELLRLEPEDPAHKPPSPHDLVYFEKSPNFCTYSGRLGTAGTAGRACNSSSPALDGCELLCCGRGHRTRTQRVTERCNCTFHWCCHVSCRNCTHTRVLHECLSMAYPYDVPDYASLGPGP

SEQ ID NO: 3 - protein, 314 aa, Homo sapiens
MGIGRSEGGRRGAALGVLLALGAALLAVGSASEYDYVSFQSDIGPYQSGRFYTKPPQCVDIPADLRLCHNVGYKKMVLPNLLEHETMAEVKQQASSWVPLLNKNCHAGTQVFLCSLFAPVCLDRPIYPCRWLCEAVRDSCEPVMQFFGFYWPEMLKCDKFPEGDVCIAMTPPNPTEASKPQGTTVCPPCDNELKSEAIIEHLCASEFALRMKIKEVKKENGDKKIVPKKKKPLKLGPIKKKDLKKLVLYLKNGADCPCHQLDNLSHHFLIMGRKVKSQYLLTAIHKWDKKNKEFKNFMKKMKNHECPTFQSVFK

SEQ ID NO: 4 - protein, 317 aa, Homo sapiens
MRAAAAAGGVRTAALALLLGALHWAPARCEEYDYYGWQAEPLHGRSYSKPPQCLDIPADLPLCHTVGYKRMRLPNLLEHESLAEVKQQASSWLPLLAKRCHSDTQVFLCSLFAPVCLDRPIYPCRSLCEAVRAGCAPLMEAYGFPWPEMLHCHKFPLDNDLCIAVQFGHLPATAPPVTKICAQCEMEHSADGLMEQMCSSDFVVKMRIKEIKIENGDRKLIGAQKKKKLLKPGPLKRKDTKRLVLHMKNGAGCPCPQLDSLAGSFLVMGRKVDGQLLLMAVYRWDKKNKEMKFAVKFMFSYPCSLYYPFFYGAAEPH

SEQ ID NO: 5 - protein, 295 aa, Homo sapiens
MLQGPGSLLLLFLASHCCLGSARGLFLFGQPDFSYKRSNCKPIPANLQLCHGIEYQNMRLPNLLGHETMKEVLEQAGAWIPLVMKQCHPDTKKFLCSLFAPVCLDDLDETIQPCHSLCVQVKDRCAPVMSAFGFPWPDMLECDRFPQDNDLCIPLASSDHLLPATEEAPKVCEACKNKNDDDNDIMETLCKNDFALKIKVKEITYINRDTKIILETKSKTIYKLNGVSERDLKKSVLWLKDSLQCTCEEMNDINAPYLVMGQKQGGELVITSVKRWQKGQREFKRISRSIRKLQC

SEQ ID NO: 6 - protein, 325 aa, Homo sapiens [sequence omitted]

SEQ ID NO: 7 - protein, 346 aa, Homo sapiens
MFLSILVALCLWLHLALGVRGAPCEAVRIPMCRHMPWNITRMPNHLHHSTQENAILAIEQYEELVDVNCSAVLRFFLCAMYAPICTLEFLHDPIKPCKSVCQRARDDCEPLMKMYNHSWPESLACDELPVYDRGVCISPEAIVTDLPEDVKWIDITPDMMVQERPLDVDCKRLSPDRCKCKKVKPTLATYLSKNYSYVIHAKIKAVQRSGCNEVTTVVDVKEIFKSSSPIPRTQVPLITNSSCQCPHILPHQDVLIMCYEWRSRMMLLENCLVEKWRDQLSKRSIQWEERLQEQRRTVQDKKKTAGRTSRSNPPKPKGKPPAPKPASPKKNIKTRSAQKRTNPKRV

SEQ ID NO: 8 - protein, 7 aa, artificial
  internal sequence
  MISC_FEATURE (1)..(1): Xaa is L or V
  MISC_FEATURE (7)..(7): Xaa is L or W
XVDGRWX

SEQ ID NO: 9 - protein, 314 aa, Homo sapiens
MGIGRSEGGRRGAALGVLLALGAALLAVGSASEYDYVSFQSDIGPYQSGRFYTKPPQCVDIPADLRLCHNVGYKKMVLPNLLEHETMAEVKQQASSWVPLLNKNCHAGTQVFLCSLFAPVCLDRPIYPCRWLCEAVRDSCEPVMQFFGFYWPEMLKCDKFPEGDVCIAMTPPNATEASKPQGTTVCPPCDNELKSEAIIEHLCASEFALRMKIKEVKKENGDKKIVPKKKKPLKLGPIKKKDLKKLVLYLKNGADCPCHQLDNLSHHFLIMGRKVKSQYLLTAIHKWDKKNKEFKNFMKKMKNHECPTFQSVFK

SEQ ID NO: 10 - DNA, 945 bp, Homo sapiens
atgggcatcgggcgcagcgaggggggccgccgcggggcagccctgggcgtgctgctggcgctgggcgcggcgcttctggccgtgggctcggccagcgagtacgactacgtgagcttccagtcggacatcggcccgtaccagagcgggcgcttctacaccaagccacctcagtgcgtggacatccccgcggacctgcggctgtgccacaacgtgggctacaagaagatggtgctgcccaacctgctggagcacgagaccatggcggaggtgaagcagcaggccagcagctgggtgcccctgctcaacaagaactgccacgccggcacccaggtcttcctctgctcgctcttcgcgcccgtctgcctggaccggcccatctacccgtgtcgctggctctgcgaggccgtgcgcgactcgtgcgagccggtcatgcagttcttcggcttctactggcccgagatgcttaagtgtgacaagttccccgagggggacgtctgcatcgccatgacgccgcccaatgccaccgaagcctccaagccccaaggcacaacggtgtgtcctccctgtgacaacgagttgaaatctgaggccatcattgaacatctctgtgccagcgagtttgcactgaggatgaaaataaaagaagtgaaaaaagaaaatggcgacaagaagattgtccccaagaagaagaagcccctgaagttggggcccatcaagaagaaggacctgaagaagcttgtgctgtacctgaagaatggggctgactgtccctgccaccagctggacaacctcagccaccacttcctcatcatgggccgcaaggtgaagagccagtacttgctgacggccatccacaagtgggacaagaaaaacaaggagttcaaaaacttcatgaagaaaatgaaaaaccatgagtgccccacctttcagtccgtgtttaagtga

SEQ ID NO: 11 - protein, 177 aa, Homo sapiens
LLEAPLLLGVRAQAAGQGPGQGPGPGQQPPPPPQQQQSGQQYNGERGISVPDHGYCQPISIPLCTDIAYNQTIMPNLLGHTNQEDAGLEVHQFYPLVKVQCSAELKFFLCSMYAPVCTVLEQALPPCRSLCERARQGCEALMNKFGFQWPDTLKCEKFPVHGAGELCVGQNTSDKGT

SEQ ID NO: 12 - protein, 87 aa, Homo sapiens
LLPAAGPAQFHGEKGISIPDHGFCQPISIPLCTDIAYNQTIMPNLLGHTNQEDAGLEVHQFYPLVKVQCSPELRFFLCSMYAPVCTV

SEQ ID NO: 13 - protein, 76 aa, Homo sapiens
FMGHIGGHSLFSCEPITLRMCQDLPYNTTFMPNLLNHYDQQTAALAMEPFHPMVNLDCSRDFRPFLCALYAPICME

SEQ ID NO: 14 - protein, 83 aa, Homo sapiens
LLLLLGPARGFGDEEERRCDPIRISMCQNLGYNVTKMPNLVGHELQTDAELQLTTFTPLIQYGCSSQLQFFLCSVYVPMCTEK

SEQ ID NO: 15 - protein, 81 aa, Homo sapiens
LAQLVGRAAAASKAPVCQEITVPMCRGIGYNLTHMPNQFNHDTQDEAGLEVHQFWPLVEIQCSPDLRFFLCTMYTPICLPD

SEQ ID NO: 16 - protein, 76 aa, Homo sapiens
FLPLLRGHSLFTCEPITVPRCMKMAYNMTFFPNLMGHYDQSIAAVEMEHFLPLANLECSPNIETFLCKAFVPTCIE

SEQ ID NO: 17 - protein, 91 aa, Homo sapiens
LLGALSAGAGAQPYHGEKGISVPDHGFCQPISIPLCTDIAYNQTILPNLLGHTNQEDAGLEVHQFYPLVKVQCSPELRFFLCSMYAPVCTV

SEQ ID NO: 18 - protein, 81 aa, Homo sapiens
LQRSSGAAAASAKELACQEITVPLCKGIGYNYTYMPNQFNHDTQDEAGLEVHQFWPLVEIQCSPDLKFFLCSMYTPICLED

SEQ ID NO: 19 - protein, 86 aa, Homo sapiens
AGGAALEIGRFDPERGRGAAPCQAVEIPMCRGIGYNLTRMPNLLGHTSQGEAAAELAEFAPLVQYGCHSHLRFFLCSLYAPMCTDQ

SEQ ID NO: 20 - protein, 85 aa, Homo sapiens
VMGSCAAISSMDMERPGDGKCQPIEIPMCKDIGYNMTRMPNLMGHENQREAAIQLHEFAPLVEYGCHGHLRFFLCSLYAPMCTEQ

SEQ ID NO: 21 - protein, 115 aa, Mus musculus
LLEAPLLLGVRAQAAGQVSGPGQQAPPPPQPQQSGQQYNGERGISIPDHGYCQPISIPLCTDMAYNQTIMPNLLGHTNQEDAGLEVHQFYPLVKVQCSAELKFFLCSMYAPVCTV

SEQ ID NO: 22 - protein, 76 aa, Mus musculus
FLGQIGGHSLFSCEPITLRMCQDLPYNTTFMPNLLNHYDQQTAALAMEPFHPMVNLDCSRDFRPFLCALYAPICME

SEQ ID NO: 23 - protein, 83 aa, Mus musculus
FLLLLRPTLGFGDEEERRCDPIRIAMCQNLGYNVTKMPNLVGHELQTDAELQLTTFTPLIQYGCSSQLQFFLCSVYVPMCTEK

SEQ ID NO: 24 - protein, 76 aa, Mus musculus
LLPLVRGHSLFTCEPITVPRCMKMTYNMTFFPNLMGHYDQGIAAVEMGHFLHLANLECSPNIEMFLCQAFIPTCTE

SEQ ID NO: 25 - protein, 91 aa, Mus musculus
LLGALPTDTRAQPYHGEKGISVPDHGFCQPISIPLCTDIAYNQTILPNLLGHTNQEDAGLEVHQFYPLVKVQCSPELRFFLCSMYAPVCTV

SEQ ID NO: 26 - protein, 81 aa, Mus musculus; identical to SEQ ID NO: 18

SEQ ID NO: 27 - protein, 86 aa, Mus musculus
TGGAALEIGRFDPERGRGPAPCQAMEIPMCRGIGYNLTRMPNLLGHTSQGEAAAQLAEFSPLVQYGCHSHLRFFLCSLYAPMCTDQ

SEQ ID NO: 28 - protein, 5 aa, artificial
  internal sequence
KKMVL

SEQ ID NO: 29 - protein, 5 aa, artificial
  internal sequence
NMTKM

SEQ ID NO: 30 - protein, 5 aa, artificial
  internal sequence
LLEHE

SEQ ID NO: 31 - protein, 5 aa, artificial
  internal sequence
HLHHS

SEQ ID NO: 32 - DNA, 75 bp, artificial
  oligonucleotide
accggtaccgggcccggaggcgggggcggagggggcggcgggggcggagggggctccaccggtcccgaattcaaa

SEQ ID NO: 33 - DNA, 18 bp, artificial
  primer
accggtaccgggcccgga

SEQ ID NO: 34 - DNA, 28 bp, artificial
  primer
tttgaattcgggaccggtggatccccct

SEQ ID NO: 35 - DNA, 21 bp, artificial
  primer
accagatctgggcccggaggc

SEQ ID NO: 36 - DNA, 38 bp, artificial
  primer
atagctagcccaccatggagccccacctgctcgggctg

SEQ ID NO: 37 - DNA, 38 bp, artificial
  primer
tttctagagttaaagcttaggccctggacccaaagaag

SEQ ID NO: 38 - DNA, 25 bp, artificial
  primer
taaaccggtgggccaggccaggggc

SEQ ID NO: 39 - DNA, 24 bp, artificial
  primer
gggccctctagactcgagtcagac

SEQ ID NO: 40 - DNA, 26 bp, artificial
  primer
gatggatccaagaccgagaagctgga

SEQ ID NO: 41 - DNA, 39 bp, artificial
  primer
atttctagaattagggtgaccagatcccagaagcccgac

SEQ ID NO: 42 - DNA, 24 bp, artificial
  primer
ggagggggatccaccggtgggcca

SEQ ID NO: 43 - DNA, 24 bp, artificial
  primer
cgcgatatccgtgcacagcgggat

SEQ ID NO: 44 - DNA, 21 bp, artificial
  primer
ctgtgcacggatatcgcgtac

SEQ ID NO: 45 - DNA, 23 bp, artificial
  primer
ccgggtagctgaagcgccgcatg

SEQ ID NO: 46 - DNA, 31 bp, artificial
  primer
acggatatcgtgaaagtgcagtgttccgctg

SEQ ID NO: 47 - protein, 6 aa, artificial
  internal sequence
  MISC_FEATURE (3)..(5): Xaa is any amino acid
KTXXXW

SEQ ID NO: 48 - protein, 4 aa, artificial
  internal sequence
ETTV

SEQ ID NO: 49 - protein, 4 aa, artificial
  internal sequence
GAAA

SEQ ID NO: 50 - protein, 6 aa, artificial
  internal sequence
  MISC_FEATURE (3)..(5): Xaa is any amino acid
AAXXXA